(19) United States
(12) Patent Application Publication
(10) Pub. No.: US 2005/0108783 A1
(43) Pub. Date: May 19, 2005

(54) Porcine invariant chain protein, full length cDNA, genomic organization, and regulatory region

(76) Inventor: Chihiro Koike, Pittsburgh, PA (US)

Correspondence Address: KING & SPALDING LLP, 191 PEACHTREE STREET, N.E., ATLANTA, GA, 30303-1763, US

(21) Appl. No.: 10/947,920

(22) Filed: Sep. 23, 2004

Related U.S. Application Data
(60) Provisional application No. 60/505,212, filed on Sep. 23, 2003.

(51) Int. Cl. A01K 67/027; C12Q 1/68; C07H 21/04; C07K 14/705

(52) U.S. Cl. 800/17; 435/69.1; 435/6; 435/320.1; 435/325; 530/350; 536/23.5

(57) ABSTRACT

The present invention provides the complete porcine invariant chain protein, full length cDNA, genomic organization, and regulatory region. Methods are provided to prepare organs, tissues, cells and animals lacking the porcine invariant chain gene for use in xenotransplantation.

### CROSS REFERENCE TO RELATED APPLICATIONS

This application claims priority to U.S. Provisional patent application Ser. No. 60/505,212.

### FIELD OF THE INVENTION

The present invention provides the complete porcine invariant chain protein, full length cDNA, genomic organization, and regulatory region. Furthermore, the present invention includes porcine animals, tissues, and organs, as well as cells and cell lines derived from such animals, tissues, and organs, which lack expression of functional porcine invariant chain protein. Such animals, tissues, organs, and cells can be used in research and in medical therapy, including in xenotransplantation. Methods are provided to prepare organs, tissues, and cells lacking the porcine invariant chain gene for use in xenotransplantation.

### BACKGROUND OF THE INVENTION

The unavailability of acceptable human donor organs, the low rate of long term success due to host versus graft rejection, and the serious risks of infection and cancer are the main challenges facing the field of tissue and organ transplantation. Because the demand for acceptable organs exceeds the supply, many people die each year while waiting for organs to become available. To help meet this demand, research has been focused on developing alternatives to allogenic transplantation. For example, dialysis has been available to patients suffering from kidney failure, artificial heart models have been tested, and other mechanical systems have been developed to assist or replace failing organs. These approaches, however, are quite expensive, and the need for frequent and periodic access to machines greatly limits the freedom and quality of life of patients undergoing such therapy.

Xenograft transplantation represents a potentially attractive alternative to artificial organs for human transplantation. The potential pool of nonhuman organs is virtually limitless, and successful xenograft transplantation would not render the patient virtually tethered to machines as is the case with artificial organ technology. Host rejection of such cross-species tissue, however, remains a major hurdle in this area, and the success of organ transplants depends on avoiding rejection of the transplant.

The forms of transplant rejection are clinically classified by their time frames and histologies. Hyperacute rejection (HAR), for example, occurs within minutes to hours following transplant. Hyperacute rejection is characterized by rapid thrombotic occlusion of the graft vasculature that begins within minutes to hours after host blood vessels are anastomosed to graft vessels. Hyperacute rejection is mediated by antibodies that pre-exist in naive hosts, the so-called ‘natural antibodies,’ which bind to endothelium and activate complement. Antibody and complement induce a number of changes in the graft endothelum that promote intravascular thrombosis. On the other hand, acute rejection typically occurs within 1-30 days, and chronic rejection occurs thereafter, sometimes taking several months to years. Some noted xenotransplants of organs from apes or old-world monkeys (e.g., baboons) into humans have been tolerated for months without rejection. However, such attempts have ultimately failed due to a number of immunological factors. Even with heavy immunosuppressive drugs used to suppress HAR, a low-grade innate immune response ultimately leads to destruction of the transplanted organs. This low grade innate immune response is attributable, in part, to failure of complement regulatory proteins (CRPs) within the graft tissue to control activation of heterologous complement on graft endothelium (see e.g., Starzl et al., Immunol. Rev., 141, 213-44 (1994)). In addition to HAR, DXR, also known as acute vascular rejection, and T-cell mediated responses also play a major role in host graft rejection. It is likely that a multifaceted strategy will need to be employed to overcome the barriers to successfully transplant non-human organs into human recipients.

Complicating the efficacy of xenotransplants further is the fact that drugs used to control innate immune responses to the xenograft can cause a non-specific depression of the immune system. Patients on such immune suppressive agents are more susceptible to the development of life-threatening infections and neoplasia.

In an effort to develop a pool of immuno-acceptable organs for xenotransplantation into humans, researchers have engineered animals producing human CRPs, an approach which has been demonstrated to delay, but not eliminate, xenograft destruction in primates (McCurry et al., Nat. Med., 1, 423-27 (1995); Bach et al., Immunol. Today, 17, 379-84 (1996)). However, organs surviving HAR may still be subjected to delayed xenograft rejection (DXR). This is characterized by the infiltration of recipient inflammatory cells and thrombosis of graft vessels, leading to ischaemia of the organ.

Whereas HAR is associated with rapid, protein-synthesis-independent, type I endothelial cell activation that results in graft rejection within minutes or hours, DXR, also known as acute vascular rejection, relates primarily to type II endothelial cell activation (see Bach F. H. et al., Immunology Today 17(8):379-384 (1996)). This response involves transcriptional induction of genes and subsequent protein synthesis resulting in the expression of adhesion molecules, cytokines, procoagulant molecules and others (Prober J. S. et al., Transplantation 50: 537-544 (1990); Prober J. S. et al., Physiol. Rev. 70: 427-451 (1990); Cotran R. S. et al., Kidney Ins. 35: 969-975 (1989)). DXR is characterized by the infiltration into the graft of host monocytes and natural killer cells (NK), which promote intragraft inflammation and thrombosis (Bach F. H. et al., Immunology Today 17(8):379-384 (1996)).

Inhibition of complement by soluble complement receptor type I (sCR1) combined with immunosuppression has been reported to delay the occurrence of DXR/AVR of porcine hearts transplanted into cynomoigus monkeys (Davis, E A et al., Transplantation 62:1018-23 (1996)). Transplantation of pig kidneys expressing human decay accelerating factor to cynomoigus monkeys also had some protective effect against DXR/AVR (Zaid A. et al., Transplantation 65:1584-90 (1998); Loss M et al., Xenotransplantation 7. 186.9 (2000)).

PCT Publication WO 02/30985A2 to Tanox Inc., teaches a method to suppress E-selectin in order to reduce DXR responses. E-selectin (also known as ELAM-1, CD62, and CD62E) is a cytokine inducible cell surface glycoprotein cell adhesion molecule that is found exclusively on endothelial cells. E-selectin mediates the adhesion of various leukocytes, including neutrophils, monocytes, eosinophils, natural killer (NK) cells, and a subset of T cells, to activated endothelium (Bevilacqua, et al., Science 243: 1160 (1989); Shimuzu, et al., Nature 349:799 (1991); Graber, et al., J. Immunol. 145: 819 (1990); Carlos, et al., Blood 77: 2266 (1991); Hakkert, et al., Blood 78:2721 (1991); and Picker, et al., Nature 349:796 (1991)). The expression of E-selectin is induced on human endothelium in response to the cytokines IL-I and TNF, as well as bacterial lipopolysaccharide (LPS), through transcriptional up-regulation (Montgomery, et al., Proc Natl Acad Sci 88:6523 (1991)). The human leukocyte receptor for human E-selectin has been identified (Berg, et al., J. Biol. Chem. 23: 14869 (1991) and Tyrrell, et al., Proc Natl Acad Sci 88:10372 (1991)). Structurally, E-selectin belongs to a family of adhesion molecules termed “selectins” that also includes P-selectin and L-selectin (see reviews in Lasky, Science 258:964 (1992) and Bevilacqua and Nelson, J. Clin. Invest. 91:379 (1993)). These molecules are characterized by common structural features such as an amino-terminal lectin-like domain, an epidermal growth factor (EGF) domain, and a discrete number of complement repeat modules (approximately 60 amino acids each) similar to those found in certain complement binding proteins. Clinically, increased E-selectin expression on endothelium is associated with a variety of acute and chronic leukocyte-mediated inflammatory reactions including allograft rejection (Allen, et al., Circulation 88: 243 (1993); Brockmeyer, et al., Transplantation 55:610 (1993); Ferran, et al Transplantation 55:605 (1993); and Taylor, et al., Transplantation 54: 451 (1992)). Studies in which the expression of human E-selectin in cardiac and renal allografts undergoing acute cellular rejection was investigated have demonstrated that E-selectin expression is selectively up-regulated in vascular endothelium of renal and cardiac tissue during acute rejection (Taylor, et al., Transplantation 54: 451 (1992)). Additionally, increased E-selectin expression correlates with the early course of cellular rejection and corresponds to the migration of inflammatory cells into the graft tissue (Taylor, et al., Transplantation 54: 451 (1992)). Taken together, these studies provide evidence that cytokine-induced expression of E-selectin by donor organ endothelium contributes to the binding and subsequent transmigration of inflammatory cells into the graft tissue and thereby plays an important role in acute cellular allograft rejection.

In addition to complement-mediated attack, human rejection of discordant xenografts appears to be mediated by a common antigen: the galactose-α(1,3)-galactose (gal-α-gal) terminal residue of many glycoproteins and glycolipids (Galili et al., Proc. Nat. Acad Sci., (USA), 84, 1369-73 (1987); Cooper, et al., Immunol. Rev., 141, 31-58 (1994); Galili, et al., Springer Sem. Immunopathol, 15, 155-171 (1993); Sandrin, et al., Transplant Rev., 8, 134 (1994)). This antigen is chemically related to the human A, B, and O blood antigens, and it is present on many parasites and infectious agents, such as bacteria and viruses. Most mammalian tissue also contains this antigen, with the notable exception of old world monkeys, apes and humans. (see, Joziasse, et al., J. Biol. Chem., 264, 14290-97 (1989). Individuals without such carbohydrate epitopes produce abundant naturally occurring antibodies (IgM as well as IgG) specific to the epitopes. Many humans show significant levels of circulating IgG with specificity for gal-a-gal carbohydrate determinants (Galili, et al., J. Exp. Med., 162, 573-82 (1985); Galili, et al., Proc. Nat. Acad. Sci. (USA), 84, 1369-73 (1987)). The α-galactosyltransferase (α-GT) enzyme catalyzes the formation of gal-α-gal moieties. Research has focused on the modulation or elimination of this enzyme to reduce or eliminate the expression of gal-α-gal moieties on the cell surface of xenotissue.

The elimination of the α-galactosyltransferase gene from porcine has long been considered one of the most significant hurdles to accomplishing xenotransplantation from pigs to humans. Two alleles in the pig genome encode the α-GT gene. Single allelic knockouts of the α-GT gene in pigs were reported in 2002 (Dai, et al. Nature Biotechnol., 20:251 (2002); Lai, et al., Science, 295:1089 (2002)).

Recently, double allelic knockouts of the α-GT gene have been accomplished (Phelps, et al., Science, 299: pp. 411-414 (2003)). WO 2004/028243 to Revivicor Inc. describes porcine animal, tissue, organ, cells and cell lines, which lack all expression of functional α1,3 galactosyltransferase (α1,3-GT). Accordingly, the animals, tissues, organs and cells lacking functional expression of α1,3-GT can be used in xenotransplantation and for other medical purposes.

PCT patent application WO 2004/016742 to Immerge Biotherapeutics, Inc. describes ζ(1,3)-galactosyltransferase null cells, methods of selecting GGTA-1 null cells, α(1,3)-galactosyltransferase null swine produced therefrom (referred to as a viable GGTA-1 null swine), methods for making such swine, and methods of using cells, tissues and organs of such a null swine for xenotransplantation.

Host T-cell mediated responses may occur against the xenograft (Sachs D. H., et al., Hum. Immunol. 28: 245-251 (1990); Sykes M. et al., Immunol. Rev. 141: 245-276 (1994)). Host T-cell stimulation depends on a complex interaction of immune effector cells and i molecules, both from the xenograft and the host. These include both CD 4+ T cell mediated responses and CD 8+ T cell mediated responses. CD4+ T-cell stimulation is normally antigen specific and tightly regulated. The foreign antigens from the xenotransplant are incorporated into the cleft of major histocompatability complex (MHC) class II molecules and transported to the cell surface, where they are displayed to CD4+ T cells. The antigenic stimulation of CD4+ Th0 cells is initiated by the cross linking of the antigen in the cleft of the MHC class II molecule on the cell surface with the T-cell receptor (TCR) on the CD4+ T cell. The MHC and antigenic complex is then presented to the CD4+ naïve T helper (Th0) cells. Initial stimulation of the T cell is antigen dependent. This reaction is enhanced by a number of co-stimulatory molecules, including the interactions of CD80 (B7.1) and CD86 (B7.2) on the presenting cell's surface with CD28 on the CD4+ Th0 cells. Furthermore, additional molecular interactions between the presenting cell and the Th0 cell takes place, involving additional signaling and enhancing adhesion (Dubey et al. 1996)). Following initial stimulation, CD4+ Th0 cells undergo division and expansion (Bird et al., 1998). Differentiation of the naïve Th0 cell is the next step in the pathway and is critical in determination of subsequent immune responses.

Differentiation of Th0 cells follows one of two pathways, developing into Th1 or Th2 cells, dependent upon a number of critical factors. CD4+ cells differentiate based on the presence of cytokine patterns. For example, IL-4 will drive CD4+ Th0 cells to differentiate into Th2 cells. Th2 cells ultimately drive humoral responses via B cell activation. Th2 cells induce the activation of B cells into plasma cells for antibody production against the specific presented antigen.

Conversely, Th0 cells may differentiate into Th1 cells. For example, IL-12/IFN-γ can drive CD4+ Th0 cells to Th1 differentiation (see Swain et al., 1996). Th1 cells are crucial in cell mediated immunity and activate CD8+ cytotoxic T lymphocytes, which can recognize antigenic proteins in the cleft of MHC class I molecules on the cell surface. Following activation by Th1 cells, CD8+ CTLs will become further activated and destroy cells displaying the antigen in the proper contex. Thus, from the above pathway, it is apparent that CD4+ T cell activation plays a significant role in generating both humoral and cell mediated responses to foreign antigens.

CD4+ T cell mediated responses are particularly strong against xenoantigens (Dorling et al., Eur J Immunol 26: 1378-1387 (1996)), and the suppression of anti-xenograft CD4+ T cell mediated responses may be one of the greatest challenges for xenotransplantation (Auchincloss, Xeno 3: 19 (1995); Auchincloss, Transplantation 46: 1 (1988)). Current methods of maintaining the level of immunosuppression required to prevent chronic xenograft rejection due to persistent CD4+ T cell mediated responses may be unfeasible using conventional systemic immunosuppressive drugs due to the increased risks of infection and neoplasia (Dorling et al., Xenotransplantation 3:112-119 (1996)). In order for xenotransplants to become an acceptable clinical alternative to allogenic organ transplants, it may be beneficial for the CD4+ T cell mediated responses to be reduced.

One approach to reduce this CD4+ T cell mediated xenograft rejection is to reduce the presentation of MHC class II molecules on the cell surface of xenograft cells, and thus reduce the stimulation of CD4+ T cells. As discussed above, the MHC class II molecules are heterodimeric complexes that display antigenic peptides on cell surfaces for display to CD4+ T cells (Unanue, Annu. Rev. Immunol. 2:395 (1984); Long, Immunol. Today 10:232 (1989); Harding et al., Cell Regul. 1:499 (1990)). MHC class II synthesis and assembly begins in the endoplasmic reticulum with the non-covalent association of the α- and β-chains with trimers of the invariant chain (Ii), also known as the γ chain (Cresswell, Annu. Rev. Immunol. 12:259 (1994)). αβ dimmers bind sequentially to a trimer of the invariant chain to form a nonameric complex which then exits the endoplasmic reticulum (Marks et. al., J. Cell Biol 111:839 (1990); Roche P et. al., Nature 354:392 (1991); Anderson et al., EMBO J. 13:675 (1994)). After being transported to the trans-golgi complex, the αβIi complex is diverted from the secretory pathway endocytic system and ultimately to acidic endosome/lysosome like structures called MHC class II compartments (Bakke and Dobberstein, Cell 63:707 (1990); Lotteau et. al., Nature 348:600 (1990); Lamb et al., Proc. Nati. Acad. Sci. USA 88:5998 (1991); Pieters et. al., J. Cell Sci. 106:831 (1993); Peters et. al., Nature 349:669 (1991); Amigorena et. al., Natuer 369:113 (1991); Qiu et. al., J. Cell Biol. 125:595 (1994); Tulp et. al., Nature 369:120 (1994); and West et al., Nature 369:147 (1994)). Once in the pathway, the luminal domain of the Ii is proteolytically degraded, leaving a small fragment, the class II-associated Ii peptide (CLIP), which is bound to the released ap dimmers. Interaction of the αβCLIP complexes in the specialized lysosome-like compartment with another class II related molecule interacts with the αβCLIP complex, removing the CLIP and allowing the αβdimers to bind peptides and display them on the cell surface of a cell. On the cell surface, the antigen loaded MHC class II molecules encounter CD4+ T cells. As discussed above, these CD4+ T-cells help further orchestrate an immune response against the antigenic peptides displayed by the class MHC complex, resulting in antibody production to the specifically recognized antigen.

The role of the invariant chain (Ii) in this presentation is very important. It essentially performs three functions in the expression of functional class II MHC molecules: 1) it helps in proper folding/assembly of MHC class II αβ dimmers; 2) it transports the newly synthesized MHC class II molecules to the endocytic compartments for peptide loading; and 3) it occupies the peptide binding groove on the MHC class II molecules during their transport from the endoplasmic reticulum to the antigen loading compartments.

The development of host immune cell reactivity to a xenograft depends on a complex interaction of immune effector molecules, most notably the recognition of the xenograft proteins as foreign. Class II MHC is actively involved in the presentation of xenogeneic antigenic peptides to a host's immune effecter cells. Disruption of Class II MHC presentation results in a reduced ability for a host to mount an immune response against a foreign protein. Cathepsins S and L play prominent roles in the degradation of the invariant chain (Ii). In I-A(b) class II mice lacking the Cathepsin S gene (Ctss) gene, failure to degrade Ii resulted in the accumulation of a class II-associated, 10-kD Ii fragment within endosomes, disrupting class II trafficking, peptide complex formation, and class II-restricted antigen presentation (Driessen et.al., J. Cell Biol. 147: 775-790, (1999)). Riese et al., Immunity 15:909-919 (2001), showed that I-A(b) class II haplotype mice lacking the Ctss gene had impaired Natural Killer 1.1 (NK1.1)+T-cell selection and function. There were no overt defects in CD4+ and CD8+ T-cell populations. Furthermore, mice deficient in the Ii protein show an inability to present native protein antigen and reduced maturation and numbers of CD4+ T cells (Viville et. al., Cell 26:635 (1993); Bikokk et. al., J. Exp. Med. 177:1699 (1993); and Elliot et. al., J. Exp. Med. 179:681 (1994); Takaesu N. T. et al., Immunity 3: 385-396 (1995)). In addition, studies suggest that invariant chain may play a role in the expression of MHC class I molecules on the cell surface (Reber A. J. et al., Immunogenetics 54(2): 74-81 (2002)).

In humans, one cause of severe combined immune deficiency syndrome is a deficiency in MHC class II molecules. This disorder, known as Bare Lymphocyte Syndrome (BLS) results in an absence of humoral and cellular immune responses to foreign antigens despite the presence of a normal number of T and B lymphocytes (Clement et al., J. Clin. Invest. 81: 669-675, (1988)). Individuals with BLS are prone to frequent bacterial, viral, protozoan, and fungal infections such as upper and lower respiratory tract infections, viral hepatitis, pseudosclerosing cholangitis, bacterial cholangitis, recurrent urinary tract infections, mucocutaneous candidiasis, meningoencephalitis, chronic lymphocytic meningitis, and poliomyelitis, amongst others. Thus, foreign proteins escape detection despite the presence of normal B and T lymphocytes due to the failure of the MHC class II molecule to properly present them to the normal immune effector cells. The clinical manifestation of BLS provides deep insights into the role MEC class II molecules play in immuno-surveillance, and the ability of foreign antigens to escape detection when these important molecules are absent from the cell surface.

A xenograft lacking a competent MHC class II molecule may be desirable for transplantation because it may be able to escape the surveillance of the host's immune effector cells by its inability to present self peptides in MHC class II complexes on its cell surface. Thus, tissues and organs from such a donor may be less antigenic, and a less antigenic donor may, therefore, produce a less vigorous immune response, regardless of the host. For example, the absence or alteration of the invariant chain in some mutant rodents results in a state of immune depression and the consequent inability of these animals to normally reject organ grafts (see, for example, Murase et al., Transplantation.55(1):l-7 (1993); Murase et al., Surgery 110:87 (19991); Murase et al., Transplant Proc 22(suppl 1):74 (1990); Lee et al., Transplant Proc 22(suppl 1):78 (1990); and Hoffman et al., Transplantation 49:483 (1990)). The Brown Norway rat is believed to have such a mutation (see, for example Murase et al. Transplantation 55 (1): pg. 6). The absence or abnormality of the invariant chain results in a reduction of the antigenic strength of tissues and organs which are therefore rejected less vigorously. Brown Norway rat livers transplanted to certain allogenic strains can permanently survive without any treatment (Murase et al., Surgery 108:880 (1990); Kamada, Experimental liver transplantation, Boca Raton, Fla.: CRC Press, 1985: 55). Heart transplants and intestinal transplants from the Brown Norway rat also show decreased rates of rejection and immuno tolerance (Guillaume et al., Surgery 91:339 (1982); Murase et al., Transplantation 55(1):1-7 (1993)). Because of this reduced immunogenicity and lack of rejection in recipient hosts, the Brown Norway rat has become a paradigm as a “universal donor” of tissues and organs from a wide spectrum of rat donor strains. The development of a human compatible “universal donor” xenograft would represent a significant advance in establishing clinically acceptable xenotransplants for humans.

The invariant chain gene has previously been identified in H. sapiens (CD74 antigen) (Claesson et. al., Proc. Natl. Acad. Sci. U.S.A. 80(24):7395-7399 (1983)), M. musculus (HG2A_MOUSE H-2 class II histocompatibility antigen) (Singer et al., EMBO J. 3(4):873-877 (1984)), B. Taurus (intracellular membrane glycoprotein type II invariant chain Ii) (Niimi et. al., Biochem. Biophys. Res. Commun. 222(l):7-12 (1996)), and R. norvegicus (CD74 antigen) (Henkes et al., Nucleic Acids Res. 16(24):11822 (1988)).

European Patent No. 0 495 852 to Imutran teaches that membrane-bound regulators of host complement expressed on the xenograft to prevent the complete activation of complement in the organ recipient. This approach has been developed and applied in order to produce transgenic animals with organs designed to survive hyperacute rejection (for example, see Squinto et. al., Curr Opin Biotech 7:641-645 (1996); McCurry et. al., Transplant Proc 28:758 (1996)). However, organs surviving HAR are subject to delayed xenograft rejection (DXR). This is characterized by the infiltration of recipient inflammatory cells and thrombosis of graft vessels, leading to ischaemia.

PCT publication No. W098/42850 to RPMS Technology Ltd.teaches that expression of coagulation inhibitors on the surface of the xenograft can inhibit the thrombotic aspect of delayed xenograft rejection (DXR).

PCT publication No. W099/57266 to Imperial College Innovators Ltd. teaches that the immunogeneic aspects of xenografts can be reduced by inhibiting T-cell mediated rejection of a xenotransplant by delivery of co-stimulatory signal 2 in order to prevent the activation of xenoreactive T-cells in the recipient.

U.S. Pat. No. 6,166,288 to Diamond et al. teaches a method of xenotransplanting organs, tissues, cells or non-viable components which reduces or prevents hyperacute rejection wherein transgenic animals are produced that express an enzyme that masks or reduces the level of the antigenic Galα(1,3)Gal or gal epitope and at least one complement inhibitor such as CD59, DAF and/or MCP.

U.S Pat. No. 6,608,030 to Plough et al. claim methods and products for suppressing a class II MHC-restricted immune response that depends upon the inhibition of invariant chain poteolysis by Cathepsin S from class II MHC/invariant chain complexes.

U.S. Pat. No. 6,100,443 to Sims et al. teaches a genetically engineered mammalian cell for transplantation into a human or animal wherein the cell does not express on its surface proteins encoded by either the class I MHC genes or the class II MRC genes which elicit a T lymphocyte mediated reaction against the cell and the cell expresses a stably incorporated nucleotide molecule which codes for a protein inhibiting complement mediated attack of the engineered cell when introduced into an animal of another species or another individual.

European Pat. No. 0591462B1 to Oklahoma Medical Research Foundation teaches the lack of expression of class II MHC on the surface of mouse cells as a result of invariant chain disruption. The disclosure is limited to the nucleotide and amino acid sequence of the murine invariant chain.

It is an object of the present invention to provide genomic and regulatory sequences of the porcine invariant chain gene.

It is an object of the present invention to provide the full length cDNA, as well as novel variants of the porcine invariant chain gene.

It is another object of the invention to provide novel nucleic acid and amino acid sequences that encode the porcine invariant chain gene.

It is yet a further object of the present invention to provide cells, tissues and/or organs deficient in the porcine invariant chain gene.

It is another object of the present invention to generate animals, particularly pigs, lacking a functional porcine invariant chain gene.

It is yet a further object of the present invention to provide cells, tissues and/or organs deficient in functional porcine invariant chain gene for use in xenotransplantation of non-human organs to human recipients in need thereof.

### SUMMARY OF THE INVENTION

The full length cDNA, peptide sequence and genomic organization of the porcine invariant chain gene have been determined. The present invention provides novel porcine invariant chain protein, cDNA, and genomic DNA regulatory sequence. Furthermore, the present invention includes porcine animals, tissues, and organs, which lack expression of a functional porcine invariant chain. Such animals, tissues, organs, and cells can be used in research and in medical therapy, including xenotransplantation. In addition, methods are provided to prepare organs, tissues, and cells lacking the porcine invariant chain gene for use in xenotransplantation. Information about the genomic organization, intronic sequences and regulatory regions of the gene are also provided.

One embodiment of the present invention provides novel nucleic acid (Table 1, Seq ID No 1) and peptide (Table 2, Seq ID No 2) sequences representing the full length cDNA encoding the porcine invariant chain. The start codon for the full length cDNA is located in the middle of exon 1, and the stop codon is located at the beginning of exon 8. Nucleotide and amino acid sequences at least 80, 85, 90, 95, 98 or 99% homologous to SEQ ID Nos 1 or 2 are provided. In addition, nucleotide and peptide sequences that contain at least 10, 15, 17, 20, 25 or 30 contiguous nucleotides or amino acids of SEQ ID Nos 1 or 2 are also provided. Further provided is any nucleotide sequence that hybridizes, optionally under stringent conditions, to SEQ ID No 1, as well as, nucleotides homologous thereto.

Another embodiment of the present invention provides nucleic acid sequences representing genomic DNA of the porcine invariant gene (Table 3, Seq ID Nos 3-18). Seq ID No. 3 represents 5′ untranslated region genomic sequence. Seq ID Nos 4-11 represent exons 1-8 respectively, and Seq ID Nos 12-18 represent introns 1-7, respectively. Nucleotide sequences at least 80, 85, 90, 95, 98 or 99% homologous to SEQ ID Nos 3-18 are provided. In addition, nucleotide and peptide sequences that contain at least 10, 15, 17, 20, 25 or 30 contigous nucleotides of SEQ ID No.s 3-18 are also provided. Further provided is any nucleotide sequence that hybridizes, optionally under stringent conditions, to SEQ ID No 3-18, as well as, nucleotides homologous thereto.

In another embodiment, the genomic sequence of the porcine invariant chain gene is represented by SEQ ID No. 19. SEQ ID NO. 19 represents a contiguous genomic sequence containing 5′ untranslated region sequence, Exon 1, Intron 1, Exon 2, Intron 2, Exon 3, Intron 3, Exon 4, Intron 4, Exon 5, Intron 5, Exon 6, Intron 6, Exon 7, Intron 7, and Exon 8 (Table 4). In addition, nucleotide sequences that contain at least 10, 15, 17, 20, 25, 30, 50, 100, 500, 650, 750, or 1000 contiguous nucleotides of SEQ ID NO. 19 are provided, as well as nucleotide sequences at least 80, 85, 90, 95, 98, or 99% homologous to SEQ ID NO. 19.

In further embodiments, nucleotide and amino acid sequences at least 80, 85, 90, 95, 98 or 99% homologous to SEQ ID Nos 1, 2, 3-18, and 19 are provided. In addition, nucleotide and peptide sequences that contain at least 10, 15, 17, 20, 25, 30, 50, 100, 150, 200, 300, 400, 500, 650, 750, 800, or 1000 contiguous nucleotide or amino acid sequences of SEQ ID Nos 1, 2, 3-18, and 19 are also provided. Further provided is any nucleotide sequence that hybridizes, optionally under stringent conditions, to SEQ ID Nos 1, 2, 3-18, and 19, as well as nucleotides homologous thereto.

Another aspect of the present invention provides nucleic acid constructs that contain cDNA or variants thereof encoding porcine invariant chain. These cDNA sequences can be derived from Seq ID Nos. 1, 2, 3-18, and 19, or any fragment thereof. Constructs can contain one, or more than one, internal ribosome entry site (IRES). The construct can also contain a promoter operably linked to the nucleic acid sequence encoding porcine invariant chain, or, alternatively, the construct can be promoterless.

In another embodiment, nucleic acid constructs are provided that contain nucleic acid sequences that permit random or targeted insertion into a host genome. The nucleic acid sequences can be derived from Seq ID Nos. 1, 2, 3-18, and 19, or any fragment thereof. In addition to the nucleic acid sequences the expression vector can contain selectable marker sequences, such as, for example, enhanced Green Fluorescent Protein (eGFP) gene sequences, initiation and/or enhancer sequences, poly A-tail sequences, and/or nucleic acid sequences that provide for the expression of the construct in prokaryotic and/or eukaryotic host cells.

In another embodiment, nucleic acid targeting vectors constructs are also provided wherein homologous recombination in somatic cells can be achieved. These targeting vectors can be transformed into mammalian cells to target the porcine invariant chain gene via homologous recombination. In one embodiment, the targeting vectors can contain a 3′ recombination arm and a 5′ recombination arm that is homologous to the genomic sequence of the porcine invariant chain gene. The homologous DNA sequence can include at least 15 bp, 20 bp, 25 bp, 50 bp, 100 bp, 500 bp, 1 kbp, 2 kbp, 4 kbp, 5 kbp, 10 kbp, 15 kbp, 20 kbp, or 50 kbp of sequence homologous to the porcine invariant chain gene sequence. In another embodiment, the homologous DNA sequence can include one or more intron and/or exon sequences.

Another embodiment of the present invention provides oligonucleotide primers capable of hybridizing to porcine invariant chain cDNA or genomic sequence, such as Seq ID Nos. 1, 3-18, or 19. In a preferred embodiment, the primers hybridize under stringent conditions to SEQ ID Nos. 1, 3-18, or 19. Another embodiment provides oligonucleotide probes capable of hybridizing to porcine invariant chain nucleic acid sequences, such as SEQ ID Nos. 1, 3-19, or 19. The polynucleotide primers or probes can have at least 14 bases, 20 bases, preferably 30 bases, or 50 bases which hybridize to a polynucleotide of the present invention. The probe or primer can be at least 14 nucleotides in length, and in a preferred embodiment, are at least 15, 20, 25, 28, or 30 nucleotides in length.

In another aspect of the present invention, mammalian cells lacking at least one allele of the porcine invariant chain gene produced according to the process, sequences and/or constructs described herein are provided. These cells can be obtained as a result of homologous recombination. Particularly, by inactivating at least one allele of the porcine invariant chain gene, cells can be produced which have reduced capability for expression of functional porcine invariant chain protein.

In embodiments of the present invention, alleles of the porcine invariant chain gene are rendered inactive according to the process, sequences and/or constructs described herein, such that the resultant porcine invariant chain is no longer generated, this reducing the functional ability of MHC Class II molecules. In one embodiment, the porcine invariant chain gene can be transcribed into RNA, but not translated into protein. In another embodiment, the porcine invariant chain gene can be transcribed in an inactive truncated form. Such a truncated RNA may either not be translated or can be translated into a nonfunctional protein. In an alternative embodiment, the porcine invariant chain gene can be inactivated in such a way that no transcription of the gene occurs. In a further embodiment, the porcine invariant chain gene can be transcribed and then translated into a nonfunctional protein.

In a further aspect of the present invention, porcine animals are provided in which at least one allele of the porcine invariant chain gene is inactivated via a genetic targeting event produced according to the process, sequences and/or constructs described herein. In another aspect of the present invention, porcine animals are provided in which both alleles of the porcine invariant chain gene are inactivated via a genetic targeting event. The gene can be targeted via homologous recombination. In other embodiments, the gene can be disrupted, i.e. a portion of the genetic code can be altered, thereby affecting transcription and/or translation of that segment of the gene. For example, disruption of a gene can occur through substitution, deletion (“knock-out”) or insertion (“knock-in”) techniques. Additional genes for a desired protein or regulatory sequence that modulate transcription of an existing sequence can be inserted.

In another aspect of the present invention, porcine cells lacking one allele, optionally both alleles of the porcine invariant chain gene can be used as donor cells for nuclear transfer into enucleated oocytes to produce cloned, transgenic animals. Alternatively, porcine invariant chain knockouts can be created in embryonic stem cells, which are then used to produce offspring. Offspring lacking a single allele of the functional invariant chain gene produced according to the process, sequences and/or constructs described herein can be breed to further produce offspring lacking functionality in both alleles through mendelian type inheritance. Cells, tissues and/or organs can be harvested from these animals for use in xenotransplantation strategies. The elimination of a functional invariant chain protein may reduce the immune rejection of the transplanted cell, tissue or organ due to the reduced capability of presenting self-antigens on the cell surface through functional MHC Class II molecules.

In one aspect of the present invention, a pig can be prepared by a method in accordance with any aspect of the present invention. Genetically modified pigs can be used as a source of tissue and/or organs for transplantation therapy. A pig embryo prepared in this manner or a cell line developed therefrom can also be used in cell-transplantation therapy. Accordingly, there is provided in a further aspect of the invention a method of therapy comprising the administration of genetically modified cells lacking porcine invariant chain to a patient, wherein the cells have been prepared from an embryo or animal lacking the porcine invariant chain gene. This aspect of the invention extends to the use of such cells in medicine, e.g. cell-transplantation therapy, and also to the use of cells derived from such embryos in the preparation of a cell or tissue graft for transplantation. The cells can be organized into tissues or organs, for example, heart, lung, liver, kidney, pancreas, corneas, nervous (e.g. brain, central nervous system, spinal cord), skin, or the cells can be islet cells, blood cells (e.g. haemocytes, i.e. red blood cells, leucocytes) or haematopoietic stem cells or other stem cells (e.g. bone marrow).

In another aspect of the present invention, porcine invariant chain-deficient pigs also lack genes encoding other xenoantigens. In one embodiment, porcine cells are provided that lack the a 1 ,3 galactosyltransferase gene, such as described in Phelps, et al., Science, 299: pp. 411414 (2003) or WO 2004/028243, and the porcine invariant chain gene produced according to the process, sequences and/or constructs described herein. In another embodiment, porcine α 1 ,3 galactosyltransferase gene knockout cells are further modified to knockout the porcine invariant chain gene produced according to the process, sequences and/or constructs described herein. In addition, porcine invariant chain deficient pigs produced according to the process, sequences and/or constructs described herein, optionally lacking one or more additional genes associated with an adverse immune response, can be modified to express complement inhibiting proteins, such as, for example, CD59, DAF, and/or MCP can be further modified to eliminate the expression of al least one allele of the porcine invariant chain gene. These animals can be used as a source of tissue and/or organs for transplantation therapy. These animals can be used as a source of tissue and/or organs for transplantation therapy. A pig embryo prepared in this manner or a cell line developed therefrom can also be used in cell-transplantation therapy.

### DESCRIPTION OF THE INVENTION

Elimination of the porcine invariant chain gene can reduce a pig organ's immunogenicity and remove an immunological barrier to xenotransplantation. The present invention is directed to novel nucleic acid sequences encoding the full-length cDNA and peptide. Information about the genomic organization, intronic sequences and regulatory regions of the gene are also provided. In one aspect, the invention provides isolated and substantially purified cDNA molecules having SEQ ID Nos: 1 or a fragment thereof. In another aspect of the invention, DNA sequences comprising the full-length genomic sequence of the porcine invariant chain gene are provided in SEQ ID Nos 3-18, and 19, or fragments thereof. In another aspect, primers for amplifying porcine invariant chain cDNA or genomic sequence derived from SEQ ID Nos. 1, 3-18, or 19, are provided. Additionally probes for identifying porcine invariant chain nucleic acid sequences derived from SEQ ID Nos. 1, 3-18, or 19, or fragments thereof are provided. DNA represented by SEQ ID Nos 3-18, or fragments thereof, can be used to construct pigs lacking functional porcine invariant chain genes. Thus, the invention also provides a porcine chromosome lacking a functional porcine invariant chain gene and a transgenic pig lacking a functional porcine invariant chain protein produced according to the process, sequences and/or constructs described herein. Such pigs can be used as tissue sources for xenotransplantation into humans. In an alternate embodiment, porcine invariant chain-deficient pigs produced according to the process, sequences and/or constructs described herein also lack other genes associated with adverse immune responses in xenotransplantation, such as, for example, the α1,3 galactosyltransferase gene. In another embodiment, pigs lacking porcine invariant chain produced according to the process, sequences and/or constructs described herein and/or other genes associated with adverse immune responses in xenotransplantation express complement inhibiting factors such as, for example, CD59, DAF, and/or MCP.

### BRIEF DESCRIPTION OF THE FIGURES

FIG. 1 provides the genomic organization of the porcine invariant chain gene. Closed bars depict each numbered exon. The length of the introns between the exons is indicated across the bottom axis labeled base pairs. The promoter region of the gene is depicted by an encircled letter P. The start codon is illustrated by a non shaded box contained within exon 1. The stop codon is depicted by a shaded box within exon 8. The class II-associated Ii peptide (CLIP) is depicted as residing within exons 3 and 4.

FIG. 2 depicts the cDNA sequence of the invariant chain gene.

FIG. 3 depicts the amino acid sequence of the invariant chain protein.

FIG. 4 illustrates representative targeting vectors, along with their corresponding genomic organization. The selectable marker genes in these particular non-limiting example are eGFP (enhanced green fluorescent protein) and the neomycin resistance (NeoR) gene. eGFP can be inserted in the DNA constructs to inactivate the porcine invariant chain gene on one allele. Alternatively, a selectable marker such as neomycin can be inserted in the DNA construct to inactivate the porcine invariant chain gene.

FIG. 5 illustrates a representative targeting vector for the “knockout” of the porcine invariant chain at the class II associated Ii peptide (CLIP). Primers generated from sequences within Exon 1 (Vf) and Exon 2 (VQ) are utilized to amplify a 5′arm for homologous recombination consisting of a portion of Exon 1, the whole of Intron 1, and Exon 2, which is cloned into a vector (iB2). Primers generated from sequences within Exon 6 (ifA) and Exon 8 (ihK) are utilized to amplify a 3′ arm for homologous recombination consisting of Exon 6, Intron 6, Exon 7, Intron 7, and a portion of Exon 8 which is cloned into a vector (i68). The iB2 vector is linearized by digestion with EcoRv and XhoI, and ligated with the i68 fragment following digestion with Eco47 III and XhoI. The target vector, when homologously recombinated with the porcine invariant chain gene, can “knock-out” Exons 3, 4, and 5, as well as Introns 2, 3, 4, and 5.

### DETAILED DESCRIPTION

Definitions.

In order to more clearly and concisely describe and disclose the subject matter of the claimed invention, the following definitions are provided for specific terms used in the specification.

A “target DNA sequence” is a DNA sequence to be modified by homologous recombination. The target DNA can be in any organelle of the animal cell including the nucleus and mitochondria and can be an intact gene, an exon or intron, a regulatory sequence or any region between genes.

A “targeting DNA sequence” is a DNA sequence containing the desired sequence modifications and which is, except for the sequence modifications, substantially isogenic with the target DNA.

A “homologous DNA sequence or homologous DNA” is a DNA sequence that is at least about 85%, 90%, 95%, 98% or 99% identical with a reference DNA sequence. A homologous sequence hybridizes under stringent conditions to the target sequence, stringent hybridization conditions include those that will allow hybridization occur if there is at least 85% and preferably at least 95% or 98% identity between the sequences.

An “isogenic or substantially isogenic DNA sequence” is a DNA sequence that is identical to or nearly identical to a reference DNA sequence. The term “substantially isogenic” refers to DNA that is at least about 97-99% identical with the reference DNA sequence, and preferably at least about 99.5-99.9% identical with the reference DNA sequence, and in certain uses 100% identical with the reference DNA sequence.

“Homologous recombination” refers to the process of DNA recombination based on sequence homology.

“Gene targeting” refers to homologous recombination between two DNA sequences, one of which is located on a chromosome and the other of which is not.

“Non-homologous or random integration” refers to any process by which DNA is integrated into the genome that does not involve homologous recombination.

A “selectable marker gene” is a gene, the expression of which allows cells containing the gene to be identified. A selectable marker can be one that allows a cell to proliferate on a medium that prevents or slows the growth of cells without the gene. Examples include antibiotic resistance genes and genes which allow an organism to grow on a selected metabolite. Alternatively, the gene can facilitate visual screening of transformants by conferring on cells a phenotype that is easily identified. Such an identifiable phenotype can be, for example, the production of luminescence or the production of a colored compound, or the production of a detectable change in the medium surrounding the cell.

The term “porcine” refers to any pig species, including pig species such as Large White, Landrace, Meishan, Minipig.

The term “oocyte” describes the mature animal ovum which is the final product of oogenesis and also the precursor forms being the oogonium, the primary oocyte and the secondary oocyte respectively.

The term “fragment” means a portion or partial sequence of a nucleotide or peptide sequence.

DNA (deoxyribonucleic acid) sequences provided herein are represented by the bases adenine (A), thymine (T), cytosine (C), and guanine(G).

Amino acid sequences provided herein are represented by the following abbreviations:

| | |
|---|---|
| A | alanine |
| P | proline |
| B | aspartate or asparagine |
| Q | glutamine |
| C | cysteine |
| R | arginine |
| D | aspartate |
| S | serine |
| E | glutamate |
| T | threonine |
| F | phenylalanine |
| G | glycine |
| V | valine |
| H | histidine |
| W | tryptophan |
| I | isoleucine |
| Y | tyrosine |
| Z | glutamate or glutamine |
| K | lysine |
| L | leucine |
| M | methionine |
| N | asparagine |

“Transfection” refers to the introduction of DNA into a host cell. Most cells do not naturally take up DNA. Thus, a variety of technical “tricks” are utilized to facilitate gene transfer. Numerous methods of transfection are known to the ordinarily skilled artisan, for example, CaPO₄ and electroporation. (J. Sambrook, E. Fritsch, T. Maniatis, Molecular Cloning: A Laboratory Manual, Cold Spring Laboratory Press, 1989). Transformation of the host cell is the indicia of successful transfection.

“Stringent conditions” refer to conditions that (1) employ low ionic strength and high temperature for washing, for example, 0.015 M NaCl/0.0015 M sodium citrate/0.1% SDS at 50° C., or (2) employ during hybridization a denaturing agent such as, for example, formamide. One skilled in the art can determine and vary the stringency conditions appropriately to obtain a clear and detectable hybridization signal. For example, stringency can generally be reduced by increasing the salt content present during hybridization and washing, reducing the temperature, or a combination thereof. See, for example, Sambrook et aL, Molecular Cloning: A Laboratory Manual, Cold Spring Harbour Laboratory Press, Cold Spring Harbour, N.Y., (1989).

### I. Complete cDNA Sequence of the Porcine Invariant Chain Gene

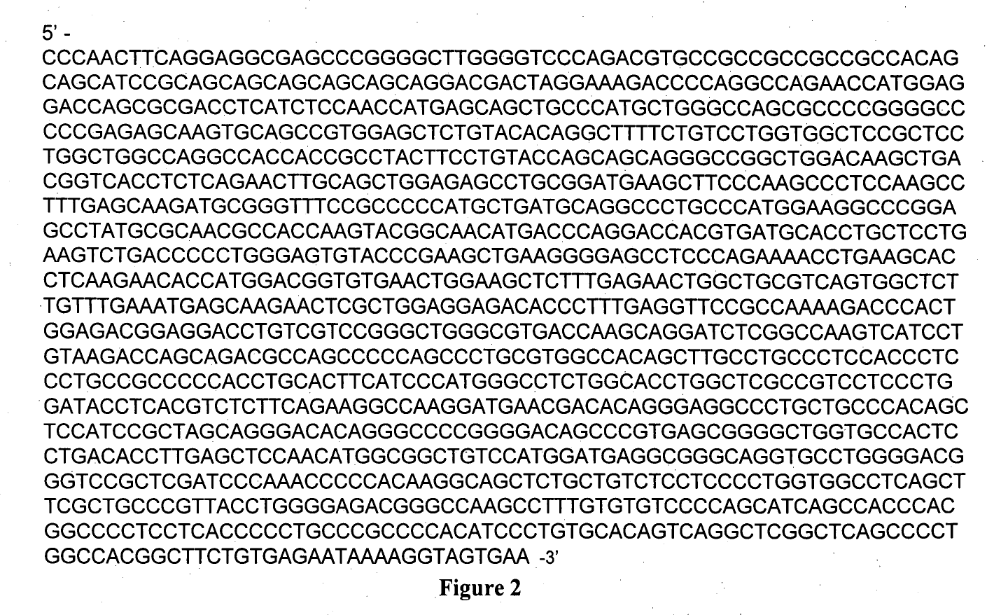

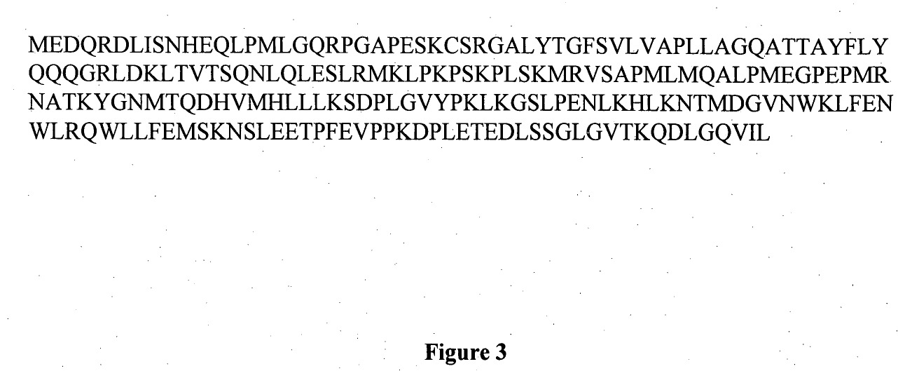

One aspect of the present invention provides novel nucleic acid (FIG. 2, Table 1, Seq ID No 1) and amino acid (FIG. 3, Table 2, Seq ID No 2) sequences representing the full-length cDNA encoding porcine invariant chain. The ATG start codon for the full-length cDNA is located at 120-122 base pairs from the beginning of exon 1. The TAA stop codon is at 9-11 base pairs from the beginning of exon 8. The polyA signal is at 16 base pairs from the end of this sequence. Nucleic and amino acid sequences at least 90, 95, 98 or 99% homologous to Seq ID Nos. 1 or 2 are provided. In addition, nucleotide and peptide sequences that contain at least 10, 15, 17, 20, 25, 30, 40, 50, 75, 100, 150, 250, 350, 500 or 1000 contiguous nucleic or amino acids of Seq ID Nos 1 or 2 are also provided. Further provided are fragments, derivatives and analogs of Seq ID Nos 1-2. Fragments of Seq ID Nos. 1-2 can include any contiguous nucleic acid or peptide sequence that includes at least about 10 bp, 15 bp, 17 bp, 20 bp, 50 bp, 100 bp, 500 bp, 1 kbp, 5 kbp or 10 kpb.

TABLE 1
FULL LENGTH cDNA OF PORCINE INVARIANT CHAIN

| | | |
|---|---|---|
| CCCAACTTCAGGAGGCGAGCCCGGGGCTTGGGGTCCC | Exons | Seq |
| AGACGTGCCGCCGCCGCCGCCACAGCAGCATCCGCAG | 1-8 | ID |
| CAGCAGCAGCAGCAGGACGACTAGGAAAGACCCCAG | | No |
| GCCAGAACCATGGAGGACCAGCGCGACCTCATCTCCA | | 1 |
| ACCATGAGCAGCTGCCCATGCTGGGCCAGCGCCCCGG | | |
| GGCCCCCGAGAGCAAGTGCAGCCGTGGAGCTCTGTAC | | |
| ACAGGCTTTTCTGTCCTGGTGGCTCCGCTCCTGGCTGG | | |
| CCAGGCCACCACCGCCTACTTCCTGTACCAGCAGCAG | | |
| GGCCGGCTGGACAAGCTGACGGTCACCTCTCAGAACT | | |
| TGCAGCTGGAGAGCCTGCGGATGAAGCTTCCCAAGCC | | |
| CTCCAAGCCTTTGAGCAAGATGCGGGTTTCCGCCCCCA | | |
| TGCTGATGCAGGCCCTGCCCATGGAAGGCCCGGAGCC | | |
| TATGCGCAACGCCACCAAGTACGGCAACATGACCCAG | | |
| GACCACGTGATGCACCTGCTCCTGAAGTCTGACCCCCT | | |
| GGGAGTGTACCCGAAGCTGAAGGGGAGCCTCCCAGAA | | |
| AACCTGAAGCACCTCAAGAACACCATGGACGGTGTGA | | |
| ACTGGAAGCTCTTTGAGAACTGGCTGCGTCAGTGGCT | | |
| CTTGTTTGAAATGAGCAAGAACTCGCTGGAGGAGACA | | |
| CCCTTTGAGGTTCCGCCAAAAGACCCACTGGAGACGG | | |
| AGGACCTGTCGTCCGGGCTGGGCGTGACCAAGCAGGA | | |
| TCTCGGCCAAGTCATCCTGTAAGACCAGCAGACGCCA | | |
| GCCCCCAGCCCTGCGTGGCCACAGCTTGCCTGCCCTCC | | |
| ACCCTCCCTGCCGCCCCCACCTGCACTTCATCCCATGG | | |
| GCCTCTGGCACCTGGCTCGCCGTCCTCCCTGGATACCT | | |
| CACGTCTCTTCAGAAGGCCAAGGATGAACGACACAGG | | |
| GAGGCCCTGCTGCCCACAGCTCCATCCGCTAGCAGGG | | |
| ACACAGGGCCCCGGGGACAGCCCGTGAGCGGGGCTG | | |
| GTGCCACTCCTGACACCTTGAGCTCCAACATGGCGGCT | | |
| GTCCATGGATGAGGCGGGCAGGTGCCTGGGGACGGGT | | |
| CCGCTCGATCCCAAACCCCCACAAGGCAGCTCTGCTG | | |
| TCTCCTCCCCTGGTGGCCTCAGCTTCGCTGCCCGTTAC | | |
| CTGGGGAGACGGGCCAAGCCTTTGTGTGTCCCCAGCA | | |
| TCAGCCACCCACGGCCCCTCCTCACCCCCTGCCCGCCC | | |
| CACATCCCTGTGCACAGTCAGGCTCGGCTCAGCCCCTG | | |
| GCCACGGCTTCTGTGAGAATAAAAGGTAGTGAA | | |

TABLE 2

FULL LENGTH AMINO ACID SEQUENCE FOR PORCINE
INVARIANT CHAIN

| | |
|---|---|
| MEDQRDLISNHEQLPMLGQRPGAPESKCSRGALYTGFSV | Seq ID No 2 |
| LVAPLLAGQATTAYFLYQQQGRLDKLTVTSQNLQLESL | |
| RMKLPKPSKPLSKMRVSAPMLMQALPMEGPEPMRNAT | |
| KYGNMTQDHVMHLLLKSDPLGVYPKLKGSLPENLKHL | |
| KNTMDGVNWKLFENWLRQWLLFEMSKNSLEETPFEVPP | |
| KDPLETEDLSSGLGVTKQDLGQVIL | |

In other aspects of the present invention, nucleic acid constructs are provided that contain cDNA or variants thereof encoding porcine invariant chain. These cDNA sequences can be SEQ ID NO 1, or derived from SEQ ID No. 2, or any fragment thereof. Constructs can contain one, or more than one, internal ribosome entry site (IRES). The construct can also contain a promoter operably linked to the nucleic acid sequence encoding porcine invariant chain, or, alternatively, the construct can be promoterless. In another embodiment, nucleic acid constructs are provided that contain nucleic acid sequences that permit random or targeted insertion into a host genome. In addition to the nucleic acid sequences the expression vector can contain selectable marker sequences, such as, for example, enhanced Green Fluorescent Protein (eGFP) gene sequences, initiation and/or enhancer sequences, poly A-tail sequences, and/or nucleic acid sequences that provide for the expression of the construct in prokaryotic and/or eukaryotic host cells. Suitable vectors and selectable markers are described below. The expression constructs can further contain sites for transcription initiation, termination, and/or ribosome binding sites. The constructs can be expressed in any prokaryotic or eukaryotic cell, including, but not limited to yeast cells, bacterial cells, such as E. coli, mammalian cells, such as CHO cells, and/or plant cells.

Promoters for use in such constructs, include, but are not limited to, the phage lambda PL promoter, E. coli lac, E. coli trp, E. coli phoA, E. coli tac prornoters, SV40 early, SV40 late, retroviral LTRs, PGKI, GALI, GALIO genes, CYCI, PH05, TRPI, ADHI, ADH2, forglymaldehyde phosphate dehydrogenase, hexokinase, pyruvate decarboxylase, phosphofructokinase, triose phosphate isomerase, phosphoglucose isomerase, glucokinase alpha-mating factor pheromone, PRBI, GUT2, GPDI promoter, metallothionein promoter, and/or mammalian viral promoters, such as those derived from adenovirus and vaccinia virus. Other promoters will be known to one skilled in the art.

### II. Genomic Sequences of the Porcine Invariant Chain Gene

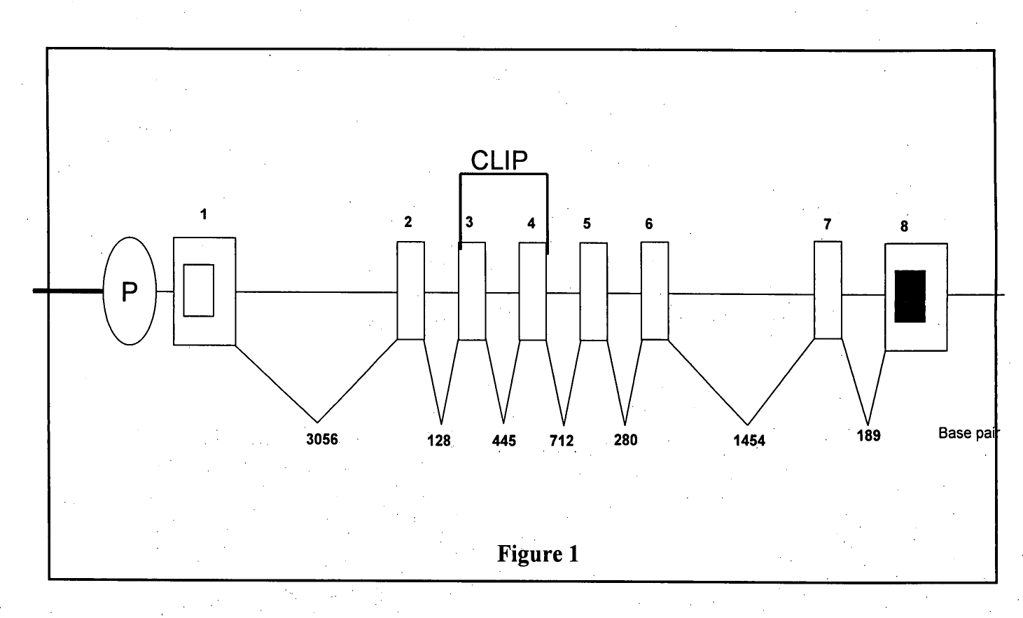

Nucleic acid sequences representing genomic DNA of the porcine invariant chain gene (FIG. 1, Table 3 and Table 4) are also provided. Seq ID Nos. 4-11-represent exons 1-8, respectively, and Seq ID Nos 12-18 represent introns 1-7, respectively. Seq. ID, No. 19. represents the 5′ UTR, Exon 1, Intron 1, Exon 2, Intron 2, Exon 3, Intron 3, Exon 4, Intron 4, Exon 5, Intron 5, Exon 6, Intron 6, Exon 7, Intron 7, and Exon 8, as well as 3′untranslated region of the genomic sequence. Nucleic and amino acid sequences at least 90, 95, 98 or 99% homologous to Seq ID Nos 4-18, and 19 are provided. In addition, nucleotide and peptide sequences that contain at least 10, 15, 17, 20 or 25, 50, 100, 150, 250, 350, 500, 1000, 2000, 2500, 5000 or 7500 contiguous nucleic acids of Seq ID Nos 4-18, and 19 are also provided. Further provided are fragments, derivatives and analogs of Seq ID Nos 4-18 and 19. Fragments of Seq ID Nos. 4-18 and 19 can include any contiguous nucleic acid or peptide sequence that includes at least about 10 bp, 15 bp, 17 bp, 20 bp, 50 bp, 100 bp, 500 bp, 1 kbp, 5 kbp or 10 kpb. Seq ID No 3 represents the 5′ untranslated region (UTR) of the gene. Nucleic acid sequences at least 90, 95, 98 or 99% homologous to Seq ID No. 3 are provided. In addition, nucleotide sequences that contain at least 10, 15, 17, 20, 25, 50, 75, 5 100 or 200 contiguous nucleic acids of Seq ID No 3 are also provided. Further provided are fragments, derivatives and analogs of Seq ID No 3. Fragments of Seq ID No. 3 can include any contiguous nucleic acid or peptide sequence that includes at least about 10 bp, 15 bp, 17 bp, 20 bp, 50 bp, 100 bp, 500 bp, 1 kbp, 5 kbp or 10 kpb.

TABLE 3
GENOMIC SEQUENCE OF PORCINE INVARIANT CHAIN GENE

| | | |
|---|---|---|
| AAACATACAAAAGACCTAAACATACAAAAGATTA | 5′ UTR | Seq |
| ATAAAGCAAGTGTGGTCTACAAGCCACAGCTGGTG | | ID |
| AGAAGTAAAGCCCTGCCAACCTCCAGGACCCTGCC | | No |
| CTGGCTTCCCTTTGCCAGGGCCTTCTCCTTCCTTCC | | 3 |
| CCGTGTAAAGTGGTGCCCCTCTCAGTGTGAGCATC | | |
| AGGAGGCAGAGGGTACAGGGTCAACATCCAGGCT | | |
| GTGATGCCCGGATGCGTGATCCTGGGCAGGCCCAG | | |
| AGCCTCTCGTCGTCTTGGCATCCCGGTCCGTAGGTG | | |
| GAGAGAAGATAAAACAGCTCTCGAAGGGGCACCG | | |
| GGAAGGTTACATGGAACAAAAAGCATGACAAGCG | | |
| ATTAGCTTGGTGCCTGGCCCGAAGGGCGGCTCGCC | | |
| AGGTGGGGGCAAGTAATGGTCGTAGAGCCTTCGAA | | |
| CCAGGGACTCGGGTTCATGAGATGCTCAAGTTAGA | | |
| CGAGAGAAGAGGGAGCGTCGACTCCAGCTTTCCTC | | |
| CCCGGCCTTTGAGAGGACAAACCCGAGCCAGAGA | | |
| GAAGCAGCGGAACTTGTCCAAGTTCCCGAAGGGGG | | |
| AGCAGCCCCTTTCTGTGCCCCCGATCTGTGAATGA | | |
| GGGGAGACAGTTTTGGTATTTTTGCAAAGCCATTTC | | |
| CTGTCCAGGGAGTGAATGTTTGCCTGTTTCTCGAGA | | |
| CCTCCACAGAGCCTTGTGAATGCAAAGGTCTACCC | | |
| GGAAACAAGTGATGAAGGCCCTGGGCAGCCAATG | | |
| GGAGCTGGCCGGCCTTTCTACCTGCCTGGGGAGTT | | |
| CCCCCTACTCCCCCTGCTCCCCTAAAGGCAGCTTTG | | |
| ACTGCGTGGGGGTATTTCCAGCTTTGTGGCTTTCAC | | |
| TTTCCACTTCTCCCAGGTGGGCGGAGTGGCCTCCTGT | | |
| GGACGAATCAGATTGCTCCTCCGC | | |
| CCCAACTTCAGGAGGCGAGCCCGGGGCTTGGGGTC | Exon 1 | Seq |
| CCAGACGTGCCGCCGCCGCCGCCACAGCAGCATCC | | ID |
| GTAGCAGCAGCAGCAGCAGGACGACTAGGAAAGA | | No |
| CCCCAGGCCAGAACCATGGAGGACCAGCGCGACCT | | 4 |
| CATCTCCAACCATGAGCAGCTGCCCATGCTGGGCC | | |
| AGCGCCCCGGGGCCCCGGAGAG | | |
| GTATGTGTGGGCGGCAGGAGGGCCACCTGGCCCTG | Intron 1 | Seq |
| GGCCCGTGGTCGTGTGCAGGGGCTTCGGATGGAGC | | ID |
| GCTAGGCTGGGAGTTAGAGGTGGTGGGGGTTCGGG | | No |
| CTCTCGCTCCAGCAGCAGCTGGCTGATTCTGAGCA | | 12 |
| GGTCCCTGGCCCACCCTGGGCTTCGGTTCTCCCTCT | | |
| TATAAAATAAGGGCCCCTCTAGGGCAGGGCTTTTA | | |
| ACTTTCTTTGCATCATGTGTTTCTCTCCTGAAAGGA | | |
| GAGCACTGGAGATTCACACAGTCCCCCTGAAGCTG | | |
| GGCCATGAACGCTAGACTAGAAATTTTGGACCAGA | | |
| TTTTGAAAATACTGTGACTCCAGGATAGAGAAGCA | | |
| AGTTCTGGAAAGAGAATGGTAGCAACAGCCATTGT | | |
| CCAGGAGGCCCCTCCGAGTTACCGCGAAGGGCAGG | | |
| CGTGTTTATGTCAAGGGAGTTGTAGCTGCAGGTGT | | |
| CCAGAGAGTAACGGAGACTTCTGCCTTCACAGGCG | | |
| GATCACGGGCGGGCAAGGGAAATGTTGCGGGGGT | | |
| CGGGAAAGGCCGAGGGGGGTCTGGCTAGATTTCTC | | |
| CAAGGGGCTGTGGGTCAGGCCCAGCCCGCACCCCT | | |
| CCCTTGCAGCAGGGGCAGCACGGCCACCGAGGGCC | | |
| CTTTCCATGTCTCCCTGAAAAGAAAGGGGGTGGTG | | |
| AGGATGCCGCTTCCCCAGTGCCCCTGCTCCCCAAA | | |
| TAATGTGCGAATCACAGGCACCTGGAAGCGTGATG | | |
| CCAGCTGTTGCCAGGCAGTCAGGGGTTTGGGAGAG | | |
| ATGCTCGGGATGTCGTGAGGAACCGCATGTGGGTA | | |
| GGTAGCTGGTGTGTCCTGTGAAAATCCTGGGGCAT | | |
| CGCGTGGCTACGAGGGCTGGATTCCAGCCCAGGTT | | |
| CGGCTGCCGCCGGGGGCGACTCGCACAGATCTGTG | | |
| CTGGAGTAGAGATCCCCATTCTCTCTGGGTCTCAG | | |
| GAGCTAGAGCAGCAGGTCCCTCTCTGTGCATCTGT | | |
| CTCTAGTCCTCCGTCACCGTGAGTGCTTCCTGTCAC | | |
| TAGTTGTGTGCTGCACATATCACGAAATGCTCAAC | | |
| TCAGTCTTAATTCTTACCGTAACCCTGTGAGTTGGG | | |
| CACTTTTATTGTTCTCAATTTAAATAAGGGAAAATT | | |
| GAGACCCTTATTAATAAGTGACTTACCTAAGTTATC | | |
| ACGGCTAGTGAATATGGGAGCCAGAATTTGAACCC | | |
| AAGACGAATCTGATTCAGGAGTTTGAATTCATTCT | | |
| ACCATCTTCGAGAGGCATGTTCTAGGGAGGGAAGG | | |
| GGGCTCCCCTCTGAGCTTGGGTCACCCCATGAGTT | | |
| GCAGACACAGTGTGGGCTCAGTGAACAGAAAGGG | | |
| TGCCCACTTATCAAATCCTAGTCTAGTTTCCTTTCA | | |
| GTTCAATCACTGCTTCAAAAAACTGACCTGGCCTG | | |
| TTTCAGACCCCTGTGTGAACACTTCTGCCTGGCAAC | | |
| ACTTGTGTCATGACTCCCGCCGGCTCTGGTTGGCTC | | |
| CTGACTTTGGCCACTGTTATTCTTACTCAAGGAGGA | | |
| AGTGAAGAGTGGGGTGACAAGAGTGGGTGCTCATC | | |
| CAGAAACAGGGGACAGGCGATCAAGACGCATAAA | | |
| GAGGAAGGAAACCAAGATGCTCAAGTTAGCTATTT | | |
| AACCCCTTGAGATGAGCGCTCGGTGTGGACGGGTG | | |
| CTTAGGGCATGGGGGCTGTGGACCCAAGAGCGCCA | | |
| CACTCCCTGACTGCATCTCATGGACCAACCTTCTCG | | |
| ATTAAGGGTTGTTCGGTGACTTGCCCCTGGTCCCCG | | |
| TGCTATCGGAGTGGCAGAGCCAGGATTTTAATCCA | | |
| GGCTACAGGATGTCCTCCAGGGCTTGGGGGAGACT | | |
| GAGGTGCAGAGACGGGATGGCGGGGACAGAGCTG | | |
| GGGCTCGACTGAACATGAGCTGCTGGAGGGGGACC | | |
| ATGAGCTGGAGCCCAGGCAAGACTCAAGCTTGGGT | | |
| GGGCTCATCACCACTTTAGAAGCCGGGCCACCCCT | | |
| GTTCTTTCAGAAGGTTGAGAAGTAGGACGTGCCTT | | |
| CCTCACCTGTGTGGTTTCTTCCTTTTCTCCACGCAG | | |
| CGTTTTGATGCTCTCCTTTCAGACTAACCATCCCTT | | |
| GGGCTTCGCCGAGGTCCAGAAATCAGAGGACCAGC | | |
| AGAAGGCCAATGAGAATGAGTCATGTCTGTGATGT | | |
| CATTAGTAACTAGGCAGAAGGGTCTCTGGCGTTT | | |
| CCCACACAGGGTGCGGTCTCACTGCAGTTTCCAGT | | |
| CGGGTCCAAGTATTCTTTCCTTGGGGTGGGTGGGA | | |
| AGAAAGCAGACAGGACCTGGGGCAGGACCCTACC | | |
| ATCCCTGTGTACATGTGCCTTCAAAACCGCAATCCC | | |
| AGGCCAAGAGCTCCATGGGCTTTTGAGATCCACCC | | |
| ACCCCTTGCAGCCAGCCAGAGAGCCCCCAAAACTC | | |
| TTCCCAGGCTCAGGCATATACGCCTGCGGAAGCCC | | |
| CCCTCCCGGTGCTCACAGTCCAGTCTTAGCTCAGA | | |
| GACCGACCTCCGAGAGACCGGGGGGAGCTGATGG | | |
| GGGCCGGCGCAAAGGCCAGAGGGGCGCAGACGTG | | |
| CTCGGAGCACAGGCAGCGTCTGGGGGAGCGTGTGT | | |
| TCCCGGGGGTGGGGCTGGTGGGGCGGCGCTGCTCC | | |
| AGTCGGACCTTGTCCAGTACCCCCAACCCATTCCTA | | |
| ACATGGCAAAGCCTCGCTTCTCTTTTTCAGGTGGGC | | |
| TGCCCGCTCTGCTGTCTGGGTCCCCCTTCCTTCAGT | | |
| CTCTGATTTCACTGCAGGGTTGGGGGGGTTGGGGC | | |
| TGAAAATCTGCTTCCCCAGAGGCGGCCTGATCCTC | | |
| AGGGCACCCTCTCCTCAACCCCTCATTGCACGGGT | | |
| GGGACTGAGGCCCTGTACCGTCCCTGGCTCTTCTCC | | |
| ACTAAGCCGAGGCCCCGGGAACCCACCAGGGCGG | | |
| CTGGAGGGAGGCTCCGGGAATGTCTTCCAGCTGCA | | |
| GGTTCACAGGCACTCACTGTGTTGTTAGGGTAGCC | | |
| AAGGGCGGGGGGCTGGGTGTGGGGGGCTCACCCC | | |
| CTCTCAGCTCACCCCCAGGGCAGCCACTTCTGCAG | | |
| TTTCTCCTCTCATCTCTCCCCTCTCGCCCCCAACAG | | |
| CAAGTGCAGCCGTGGAGCTCTGTACACAGGCTTTT | Exon 2 | Seq |
| CTGTCCTGGTGGCTCCGCTCCTGGCTGGCCAGGCC | | ID |
| ACCACCGCCTACTTCCTGTACCAGCAGCAGGGCCG | | No |
| GCTGGACAAGCTGACGGTCACCTCTCAGAACTTGC | | 5 |
| AGCTGGAGAGCCTGCGGATGAAGCTTCCCAAGC | | |
| GTGCGTGCCCCACCCCCACTCCTCCCCCTCTGCTCT | Intron 2 | Seq |
| GGAACCCTGGCCCCTGCCCTGTTGGGGGGGGCGGC | | ID |
| TTGGTCCTTCTTCCCTGGGCATAGGCCACACTCACG | | No |
| CTGCCCCTCCCGCTCCCACAG | | 13 |
| CCTCCAAGCCTTTGAGCAAGATGCGGGTTTCCGCC | Exon 3 | Seq |
| CCCATGCTGATGCAGGCCCTGCCCATGGAAGGCCC | | ID |
| GGAG | | No |
| | | 6 |
| GTACGGCCGGACAGGCCGGGGCGGGGGGCGGGGG | Intron 3 | Seq |
| CACCGCGGGCCCTGGCAGAGCACAGCTGGGGAGG | | ID |
| GGCTCCTGACCGCGGGGGAGGAAGGGGCGCTCCG | | No |
| GGAGTCGGGAAGGCGAGAGCCCCCTGGGCCCCTG | | 14 |
| GGACGGGGGCTCAGAGTAGCACAGCTTCCCAGAGC | | |
| GCCTCCGTCCCTTCTCGGCCCTGGTGTCTATCAAAC | | |
| GTGGGCAACGTCTGTTCTCGAGACTGCCTCCTGTCC | | |
| ACGAAGCCTTGGGCGGGAAAGCGGGTGCCTATGTG | | |
| ACAGAAGCACCAAGGCGGGCGAGTCGTGCCGCGC | | |
| CAGAGCTCTCCCCTCTGGAGGCAGCAGGGCGGGCC | | |
| CGGGGAACATCTGGGAAGCCTGCTGCTGGCTCTGC | | |
| CCCGGGCCTGGCCTGCAGGGCCGTGCTGGTAACCA | | |
| CCTGCCCCCAATCCCTCCTTCTCCGCAG | | |
| CCTATGCGCAACGCCACCAAGTACGGCAACATGAC | Exon 4 | Seq |
| CCAGGACCACGTGATGCACCTGCTCCTG | | ID |
| | | No |
| | | 7 |
| GTGAGTGAGCGGGGCCAGGCAGGCCTGGCCTCCCA | Intron 4 | Seq |
| GCCCACCCCCACGGGCCCCAGGCTGCGCGCCAGGA | | ID |
| CTGGTACCCGCCTGACTGGTGCAGACACGCGGAAG | | No |
| GAGCTCGCTGGCTTTGGGAGGAAAGGCGAGCGTGG | | 15 |
| CCCTCAGCGTTCTGCAGAGTCCAGCCTCCACTGCCC | | |
| GCATCCCTGTGTCTGAGCCAAGAAACTATGGCCTG | | |
| AAGCCTGGAAGTGACTCGCTCAGGATGCCCGCTTT | | |
| TCCCTCCCGAGGGTTTAGTGCGCGTCTTCCCAGTGC | | |
| CAGGTGTCGTGCGAGGCTCCAGGCCCCCCCAGTGA | | |
| GGCAGTCAGACCCCAGCTCCCCGTCCCAGGGGGAC | | |
| CCCAGGCCAGCCCGAGAGGGGTGGGCGACCGGGT | | |
| GAGGGGGCTGTGCAAAGGGCCCAGCCTCCCGGAG | | |
| GGACTGGATGAGGTGGGGGAGGAGCTCCTGGAGG | | |
| AAGCAGACCCGAGGGACGCAGAGGAGCCTGCCAG | | |
| CAGGAGGAGAGCTGGGGTCGTTGACAGCACCGGG | | |
| TCCGCAGACTCCCAGCCCTGCGGTCCCGTAGCTGA | | |
| GCGGCTGGACCCCTCCGCCCTCCTCCCAGCAGCTC | | |
| CGGCACCCGAGGCCCTGGGCCCATCCCTCAGCAGA | | |
| CGGGGGGCCTGGGGGCCCGGGCGGCGCGTGAACC | | |
| CTGCCTGCCTGGCAGCCCTTTAACCCTCCTTCCCGC | | |
| CTTGCGACCCTGCAG | | |
| AAGTCTGACCCCCTGGGAGTGTACCCGAAGCTGAA | Exon 5 | Seq |
| GGGGAGCCTCCCAGAAAACCTGAAGCACCTCAAG | | ID |
| AACACCATGGACGGTGTGAACTGGAAG | | No |
| | | 8 |
| GTCAGCAGCGGCCCCGCACCGGGGGCCCTCCCTCC | Intron 5 | Seq |
| CGATGCCCAGAGCAGCCCCGGAGAGCTGGGCTAA | | ID |
| GCGGGGAAGCCACCTGCAGGGAGCTGGAGGCGGC | | No |
| CTTGCCAGCATCTCCCAGGGCCGGAGTGGCAGGGT | | 16 |
| GCCTGGAGGCGAGGAGCCGTGCGGCCAAGCCCGT | | |
| GGGCCTGAGTGGGCGCCAGGGCCCGCTGGTGCCCC | | |
| GTGTCCGTCCCCAGGGGCGAGAGCGGGCGGGGAG | | |
| GGGCTGCTGGGCCTGACCCTCTGCCTTTATCCCTCT | | |
| CAG | | |
| CTCTTTGAGAACTGGCTGCGTCAGTGGCTCTTGTTT | Exon 6 | Seq |
| GAAATGAGCAAGAACTCGCTGGAGGAGACACCCTT | | ID |
| TGAGGTTCCGCCAAAAG | | No |
| | | 9 |
| GTAGAGAATTGGTGCCGGGGTGGGGTGGGGTGGG | Intron 6 | Seq |
| GATGCGGCGTTTCTGGGCGCCCGGTCCACGCACAG | | ID |
| CGCTCTCGGCCTCTCGGCTCTCACTCTGGCTCCCCT | | No |
| GCCTGGTCTCCTGGGCCGACTGCCCCCCGCCCCGC | | 17 |
| GGGGGATGCTCCTGCAGAGGACCCAGGACTTCCCA | | |
| GGGGTCACGGCAGGGACCGTCCCCGTCCAGCACTG | | |
| CCTCCCGCAGGGTCCCATGAGGGGGCTGGGCTCAT | | |
| GACCCGGCCCCTCGGCGGGGGGCACCTCACACAGC | | |
| CAGCAGGCTCCAGCGCGGAGGGCCCGGAGTTTCCC | | |
| GGCTCACGGGCAGCGTGTACCAGCCTCTCCAGCCC | | |
| CGACGCCTTTGCGAGTTGGGTTTTTATCCCGGAGCT | | |
| GATGGGCTGTGCACGTGGACCTGTCTCTTCCCTCTG | | |
| GGCTGCGGTTTTAGAGGTGGGGGACCCACGGCCCA | | |
| CCCTCCCTGGGCCTCTGATCCCCGCATCAGATTCCC | | |
| AGAGGGCACCCTGGGCGCTGTCTCCCCCACTGCCA | | |
| GCCAGCGTCTGCCTCCCTCCCAGGAGCCCTCACAT | | |
| GGCACCCGCTTCTCCCTGGATACTCTGGTGAGGGA | | |
| AGCCCAAACTCGCCATGATGCCCACAGCTTTTGTCT | | |
| AGAAATCGGAATTTCGGGCGATGCTAGACTCCAGC | | |
| GGGCCCTGGGCGTCAGAAGGAATCTCATTCTCGAA | | |
| CGAGCCGCACGGCCGTTCGCTTCCTCTTGGGTCCG | | |
| GGACCTGGGCCCAGGCCTGTTTCCCCGTCTGCGCC | | |
| GGGGGTAGTAGGACAGGGCCACCTGAGCAGAGGG | | |
| CGGAGGCCCCAGCAGTGGTGCGTGGGCTCTGGTTC | | |
| TGAGACCCTGGCCCAGAGCCGCTCGCTCACTGCAG | | |
| TCGCCGTGCCTGTCGCTCCAGTACTGACCAAGTGC | | |
| CAGGAAGAGGTCAGCCGTGTCCCCGCCGTCCACCC | | |
| GGGCATGTTCAGGCCCAAGTGCGACGAGGACGGC | | |
| AACTACATGCCGCTCCAGTGCTACGGGAGCATCGG | | |
| CTTCTGCTGGTGCGTCTTCCCCAACGGCACCGAGGT | | |
| CCCGCACACCAGGAGCCGCGGGCGCCACAGCTGCA | | |
| GCGGTAAGCGGTGGCCTCGGCGCCAGGAGCAGGA | | |
| GGGCCCAGGGCCAGGACGGCAAGGCGGCCACTCC | | |
| TGGGGGCAGCCTCTGGCTGTACGCCCACCTGTCTG | | |
| CCCCTCACATCCGCACTCCTGTCTGATCCGATCTGT | | |
| ACCCGTCAAAGCGCCGCCCGGCCACGCAGCCAGGC | | |
| CTCCAGACCCGCGTCCTTCCTCACTCACTTCCCGGC | | |
| CACACTGCACGGCCCCTGCCTGCCATCCGGCAAGC | | |
| ATCCCTGCTTGGCCAGGCGCCGGGCCTGCGGCGAG | | |
| ATGGGCTAGAACGAGCCCACCCAGGCCCCTGCCAG | | |
| CGGTGCCCCAGGCTGGCCTGCGCTGACAGGGGACC | | |
| CTCTGCTCCTTTGCAG | | |
| ACCCACTGGAGACGGAGGACCTGTCGTCCGGGCTG | Exon 7 | Seq |
| GGCGTGACCAAGCAGGATCTCGGCCAAG | | ID |
| | | No |
| | | 10 |
| GTAAGGGCCCTGCCTCCCAGCAGCCCAACCCCAGG | Intron 7 | Seq |
| GGGGCTGACGTCCCCCAGCACCCATCTGGCGGAGG | | ID |
| AGGCCGGGGGCTGGGGGTCTCAGGGGCTCCGACA | | No |
| GGCAGGCCTTGTCCTCACGTAGGAGGGTGAGGCCA | | 18 |
| CAGGGTGCTGGGGCGGGGGGAGCAGGGACAGTAG | | |
| GGGACCCCAACCCTAAGCCACTGCCCCTCTCTCCA | | |
| G | | |
| TCATCCTGTAAGACCAGCAGACGCCAGCCCCCAGC | Exon 8 | Seq |
| CCTGCGTGGCCACAGCTTGCCTGCCCTCCACCCTCC | | ID |
| CTGCCGCCCCCACCTGCACTTCATCCCATGGGCCTC | | No |
| TGGCACCTGGCTCGCCGTCCTCCCTGGATACCTCAC | | 11 |
| GTCTCTTCAGAAGGCCAAGGATGAACGACACAGGG | | |
| AGGCCCTGCTGCCCACAGCTCCATCCGCTAGCAGG | | |
| GACACAGGGCCCCGGGGACAGCCCGTGAGCGGGG | | |
| CTGGTGCCACTCCTGACACCTTGAGCTCCAACATG | | |
| GCGGCTGTCCATGGATGAGGCGGGCAGGTGCCTGG | | |
| GGACGGGTCCGCTCGATCCCAAACCCCCACAAGGA | | |
| CAGCTCTGCTGTCTCCTCCCCTGGTGGCCTCAGCTT | | |
| CGCTGCCCGTTACCTGGGGAGACGGGCCAAGCCTT | | |
| TGTGTGTCCCCAGCATCAGCCACCCACGGCCCCTC | | |
| CTCACCCCCTGCCCGCCCCACATCCCTGTGCACAGT | | |
| CAGGCTCGGCTCAGCCCCTGGCCACGGCTTCTGTG | | |
| AGAATAAAAGGTAGTGAATTAGGAC | | |

TABLE 4
GENOMIC SEQUENCE OF PORCINE INVARIANT CHAIN GENE

| | |
|---|---|
| AAACATACAAAAGACCTAAACATACAAAAGATTAATAAAGCAAGT | Seq. ID. |
| GTGGTCTACAAGCCACAGCTGGTGAGAAGTAAAGCCCTGCCAACCT | No. 19 |
| CCAGGACCCTGCCCTGGCTTCCCTTTGCCAGGGCCTTCTCCTTCCTT | |
| CCCCGTGTAAAGTGGTGCCCCTCTCAGTGTGAGCATCAGGAGGCAG | |
| AGGGTACAGGGTCAACATCCAGGCTGTGATGCCCGGATGCGTGATC | |
| CTGGGCAGGCCCAGAGCCTCTCGTCGTCTTGGCATCCCGGTCCGTA | |
| GGTGGAGAGAAGATAAAACAGCTCTCGAAGGGGCACCGGGAAGGT | |
| TACATGGAACAAAAAGCATGACAAGCGATTAGCTTGGTGCCTGGCC | |
| CGAAGGGCGGCTCGCCAGGTGGGGGCAAGTAATGGTCGTAGAGCC | |
| TTCGAACCAGGGACTCGGGTTCATGAGATGCTCAAGTTAGACGAGA | |
| GAAGAGGGAGCGTCGACTCCAGCTTTCCTCCCCGGCCTTTGAGAGG | |
| ACAAACCCGAGCCAGAGAGAAGCAGCGGAACTTGTCCAAGTTCCC | |
| GAAGGGGGAGCAGCCCCTTTCTGTGCCCCCGATCTGTGAATGAGGG | |
| GAGACAGTTTTGGTATTTTTGCAAAGCCATTTCCTGTCCAGGGAGTG | |
| AATGTTTTGCCTGTTTCTCGAGACCTCCACAGAGCCTTGTGAATGCAA | |
| AGGTCTACCCGGAAACAAGTGATGAAGGCCCTGGGCAGCCAATGG | |
| GAGCTGGCCGGCCTTTCTACCTGCCTGGGGAGTTCCCCCTACTCCCC | |
| CTGCTCCCCTAAAGGCAGCTTTGACTGCGTGGGGGTATTTCCAGCTT | |
| TGTGGCTTTCACTTCCACTTCTCCCAGGTGGGCGGAGTGGCCTCCTG | |
| TGGACGAATCAGATTGCTCCTCCGCCCCAACTTCAGGAGGCGAGCC | |
| CGGGGCTTGGGGTCCCAGACGTGCCGCCGCCGCCGCCACAGCAGCA | |
| TCCGTAGCAGCAGCAGCAGCAGGACGACTAGGAAAGACCCCAGGC | |
| CAGAACCATGGAGGACCAGCGCGACCTCATCTCCAACCATGAGCAG | |
| CTGCCCATGCTGGGCCAGCGCCCCGGGGCCCCGGAGAGGTATGTGT | |
| GGGCGGCAGGAGGGCCACCTGGCCCTGGGCCCGTGGTCGTGTGCAG | |
| GGGCTTCGGATGGAGCGCTAGGCTGGGAGTTAGAGGTGGTGGGGG | |
| TTCGGGCTCTCGCTCCAGCAGCAGCTGGCTGATTCTGAGCAGGTCC | |
| CTGGCCCACCCTGGGCTTCGGTTCTCCCTCTTATAAAATAAGGGCCC | |
| CTCTAGGGCAGGGCTTTTAACTTTCTTTGCATCATGTGTTTCTCTCCT | |
| GAAAGGAGAGCACTGGAGATTCACACAGTCCCCCTGAAGCTGGGC | |
| CATGAACCCTAGACTAGAAATTTTGGACCAGATTTTGAAAATACTG | |
| TGACTCCAGGATAGAGAAGCAAGTTCTGGAAAGAGAATGGTAGCA | |
| ACAGCCATTGTCCAGGAGGCCCCTCCGAGTTACCGCGAAGGGCAGG | |
| CGTGTTTATGTCAAGGGAGTTGTAGCTGCAGGTGTCCAGAGAGTAA | |
| CGGAGACTTCTGCCTTGACAGGCGGATCACGGGCGGGCAAGGGAA | |
| ATGTTGCGGGGGTCGGGAAAGGCCGAGGGGGGTCTGGCTAGATTTC | |
| TCCAAGGGGCTGTGGGTCAGGCCCAGCCCGCACCCCTCCCTTGCAG | |
| CAGGGGCAGCACGGCCACCGAGGGCCCTTTCCATGTCTCCCTGAAA | |
| AGAAAGGGGGTGGTGAGGATGCCGCTTCCCCAGTGCCCCTGCTCCC | |
| CAAATAATGTGCGAATCACAGGCACCTGGAAGCGTGATGCCAGCTG | |
| TTGCCAGGCAGTCAGGGGTTTGGGAGAGATGCTCGGGATGTCGTGA | |
| GGAACCGCATGTGGGTAGGTAGCTGGTGTGTCCTGTGAAAATCCTG | |
| GGGCATCGCGTGGCTACGAGGGCTGGATTCCAGCCCAGGTTCGGCT | |
| GCCGCCGGGGGCGACTCGCACAGATCTGTGCTGGAGTAGAGATCCC | |
| CATTCTCTCTGGGTCTCAGGAGCTAGAGCAGCAGGTCCCTCTCTGTG | |
| CATCTGTCTCTAGTCCTCCGTCACCGTGAGTGCTTCCTGTCACTAGT | |
| TGTGTGCTGCACATATCACGAAATGCTCAACTCAGTCTTAATTCTTA | |
| CCGTAACCCTGTGAGTTGGGCACTTTTATTGTTCTCAATTTAAATAA | |
| GGGAAAATTGAGACCCTTATTAATAAGTGACTTACCTAAGTTATCA | |
| CGGCTAGTGAATATGGGAGCCAGAATTTGAACCCAAGACGAATCTG | |
| ATTCAGGAGTTTGAATTCATTCTACCATCTTCGAGAGGCATGTTCTA | |
| GGGAGGGAAGGGGGCTCCCCTCTGAGCTTGGGTCACCCCATGAGTT | |
| GCAGACACAGTGTGGGCTCAGTGAACAGAAAGGGTGCCCACTTATC | |
| AAATCCTAGTCTAGTTTCCTTTCAGTTCAATCACTGCTTCAAAAAAC | |
| TGACCTGGCCTGTTTCAGACCCCTGTGTGAACACTTCTGCCTGGCAA | |
| CACTTGTGTCATGACTCCCGCCGGCTCTGGTTGGCTCCTGACTTTGG | |
| CCACTGTTATTCTTACTCAAGGAGGAAGTGAAGAGTGGGGTGACAA | |
| GAGTGGGTGCTCATCCAGAAACAGGGGACAGGCGATCAAGACGCA | |
| TAAAGAGGAAGGAAACCAAGATGCTCAAGTTAGCTATTTAACCCCT | |
| TGAGATGAGCGCTCGGTGTGGACGGGTGCTTAGGGCATGGGGGCTG | |
| TGGACCCAAGAGCGCCACACTCCCTGACTGCATCTCATGGACCAAC | |
| CTTCTCGATTAAGGGTTGTTCGGTGACTTGCCCCTGGTCCCCGTGCT | |
| ATCGGAGTGGCAGAGCCAGGATTTTAATCCAGGCTACAGGATGTCC | |
| TCCAGGCCTTGGGGGAGACTGAGGTGCAGAGACGGGATGGCGGGG | |
| ACAGAGCTGGGGCTCGACTGAACATGAGCTGCTGGAGGGGGACCA | |
| TGAGCTGGAGCCCAGGCAAGACTCAAGCTTGGGTGGGCTCATCACC | |
| ACTTTAGAAGCCGGGCCACCCCTGTTCTTTCAGAAGGTTGAGAAGT | |
| AGGACGTGCCTTCCTCACCTGTGTGGTTTCTTCCTTTTCTCCACGCA | |
| GCGTTTTGATGCTCTCCTTTCAGACTAACCATCCCTTGGGCTTCGCC | |
| GAGGTCCAGAAATCAGAGGACCAGCAGAAGGCCAATGAGAATGAG | |
| TCATGTCTGTGATGTCATTAGTAACTAGGCAGAAGGGTCTTCTGGC | |
| GTTTCCCACACAGGGTGCGGTCTCACTGCAGTTTCCAGTCGGGTCCA | |
| AGTATTCTTTCCTTGGGGTGGGTGGGAAGAAAGCAGACAGGACCTG | |
| GGGCAGGACCCTACCATCCCTGTGTACATGTGCCTTCAAAACCGCA | |
| ATCCCAGGCCAAGAGCTCCATGGGCTTTTGAGATCCACCCACCCCT | |
| TGCAGCCAGCCAGAGAGCCCCCAAAACTCTTCCCAGGCTCAGGCAT | |
| ATACGCCTGCGGAAGCCCCCCTCCCGGTGCTCACAGTCCAGTCTTA | |
| GCTCAGAGACCGACCTCCGAGAGACCGGGGGGAGCTGATGGGGGC | |
| CGGCGCAAAGGCCAGAGGGGCGCAGACGTGCTCGGAGCACAGGCA | |
| GCGTCTGGGGGAGCGTGTGTTCCCGGGGGTGGGGCTGGTGGGGCGG | |
| CGCTGCTCCAGTCGGACCTTGTCCAGTACCCCCAACCCATTCCTAAC | |
| ATGGCAAAGCCTCGCTTCTCTTTTTCAGGTGGGCTGCCCGCTCTGCT | |
| GTCTGGGTCCCCCTTCCTTCAGTCTCTGATTTCACTGCAGGGTTGGG | |
| GGGGTTGGGGCTGAAAATCTGCTTCCCCAGAGGCGGCCTGATCCTC | |
| AGGGCACCCTCTCCTCAACCCCTCATTGCACGGGTGGGACTGAGGC | |
| CCTGTACCGTCCCTGGCTCTTCTCCACTAAGCCGAGGCCCCGGGAA | |
| CCCACCAGGGCGGCTGGAGGGAGGCTCCGGGAATGTCTTCCAGCTG | |
| CAGGTTCACAGGCACTCACTGTGTTGTTAGGGTAGCCAAGGGCGGG | |
| GGGCTGGGTGTGGGGGGCTCACCCCCTCTCAGCTCACCCCCAGGGC | |
| AGCCACTTCTGCAGTTTCTCCTCTCATCTCTCCCCTCTCGCCCCCAAC | |
| AGCAAGTGCAGCCGTGGAGCTCTGTACACAGGCTTTTCTGTCCTGG | |
| TGGCTCCGCTCCTGGCTGGCCAGGCCACCACCGCCTACTTCCTGTAC | |
| CAGCAGCAGGGCCGGCTGGACAAGCTGACGGTCACCTCTCAGAACT | |
| TGCAGCTGGAGAGCCTGCGGATGAAGCTTCCCAAGCGTGCGTGCCC | |
| CACCCCCACTCCTCCCCCTCTGCTCTGGAACCCTGGCCCCTGCCCTG | |
| TTGGGGGGGGCGGCTTGGTCCTTCTTCCCTGGGCATAGGCCACACT | |
| CACGCTGCCCCTCCCGCTCCCACAGCCTCCAAGCCTTTGAGCAAGA | |
| TGCGGGTTTCCGCCCCCATGCTGATGCAGGCCCTGCCCATGGAAGG | |
| CCCGGAGGTACGGCCGGACAGGCCGGGGCGGGGGGCGGGGGCACC | |
| GCGGGCCCTGGCAGAGCACAGCTGGGGAGGGGCTCCTGACCGCGG | |
| GGGAGGAAGGGGCGCTCCGGGAGTCGGGAAGGCGAGAGCCCCCTG | |
| GGCCCCTGGGACGGGGGCTCAGAGTAGCACAGCTTCCCAGAGCGCC | |
| TCCGTCCCTTCTCGGCCCTGGTGTCTATCAAACGTGGGCAACGTCTG | |
| TTCTCGAGACTGCCTCCTGTCCACGAAGCCTTGGGCGGGAAAGCGG | |
| GTGCCTATGTGACAGAAGCACCAAGGCGGGCGAGTCGTGCCGCGCC | |
| AGAGCTCTCCCCTCTGGAGGCAGCAGGGCGGGCCCGGGGAACATCT | |
| GGGAAGCCTGCTGCTGGCTCTGCCCCGGGCCTGGCCTGCAGGGCCG | |
| TGCTGGTAACCACCTGCCCCCAATCCCTCCTTCTCCGCAGCCTATGC | |
| GCAACGCCACCAAGTACGGCAACATGACCCAGGACCACGTGATGC | |
| ACCTGCTCCTGGTGAGTGAGCGGGGCCAGGCAGGCCTGGCCTCCCA | |
| GCCCACCCCCACGGGCCCCAGGCTGCGCGCCAGGACTGGTACCCGC | |
| CTGACTGGTGCAGACACGCGGAAGGAGCTCGCTGGCTTTGGGAGGA | |
| AAGGCGAGCGTGGCCCTCAGCGTTCTGCAGAGTCCAGCCTCCACTG | |
| CCCGCATCCCTGTGTCTGAGCCAAGAAACTATGGCCTGAAGCCTGG | |
| AAGTGACTCGCTCAGGATGCCCGCTTTTCCCTCCCGAGGGTTTTAGTG | |
| CGCGTCTTCCCAGTGCCAGGTGTCGTGCGAGGCTCCAGGCCCCCCC | |
| AGTGAGGCAGTCAGACCCCAGCTCCCCGTCCCAGGGGGACCCCAGG | |
| CCAGCCCGAGAGGGGTGGGCGACCGGGTGAGGGGGCTGTGCAAAG | |
| GGCCCAGCCTCCCGGAGGGACTGGATGAGGTGGGGGAGGAGCTCC | |
| TGGAGGAAGCAGACCCGAGGGACGCAGAGGAGCCTGCCAGCAGGA | |
| GGAGAGCTGGGGTCGTTGACAGCACCGGGTCCGCAGACTCCCAGCC | |
| CTGCGGTCCCGTAGCTGAGCGGCTGGACCCCTCCGCCCTCCTCCCA | |
| GCAGCTCCGGCACCCGAGGCCCTGGGCCCATCCCTCAGCAGACGGG | |
| GGGCCTGGGGGCCCGGGCGGCGCGTGAACCCTGCCTGCCTGGCAGC | |
| CCTTTAACCCTCCTTCCCGCCTTGCGACCCTGCAGAAGTCTGACCCC | |
| CTGGGAGTGTACCCGAAGCTGAAGGGGAGCCTCCCAGAAAACCTG | |
| AAGCACCTCAAGAACACCATGGACGGTGTGAACTGGAAGGTCAGC | |
| AGCGGCCCCGCACCGGGGGCCCTCCCTCCCGATGCCCAGAGCAGCC | |
| CCGGAGAGCTGGGCTAAGCGGGGAAGCCACCTGCAGGGAGCTGGA | |
| GGCGGCCTTGCCAGCATCTCCCAGGGCCGGAGTGGCAGGGTGCCTG | |
| GAGGCGAGGAGCCGTGCGGCCAAGCCCGTGGGCCTGAGTGGGCGC | |
| CAGGGCCCGCTGGTGCCCCGTGTCCGTCCCCAGGGGCGAGAGCGGG | |
| CGGGGAGGGGCTGCTGGGCCTGACCCTCTGCCTTTATCCCTCTCAGC | |
| TCTTTGAGAACTGGCTGCGTCAGTGGCTCTTGTTTTGAAATGAGCAA | |
| GAACTCGCTGGAGGAGACACCCTTTGAGGTTCCGCCAAAAGGTAGA | |
| GAATTGGTGCCGGGGTGGGGTGGGGTGGGGATGCGGCGTTTCTGGG | |
| CGCCCGGTCCACGCACAGCGCTCTCGGCCTCTCGGCTCTCACTCTGG | |
| CTCCCCTGCCTGGTCTCCTGGGGCGACTGCCCCCCGCCCCGCGGGG | |
| GATGCTCCTGCAGAGGACCCAGGACTTCCCAGGGGTCACGGCAGGG | |
| ACCGTCCCCGTCCAGCACTGCCTCCCGCAGGGTCCCATGAGGGGGC | |
| TGGGCTCATGACCCGGCCCCTCGGCGGGGGGCACCTCACACAGCCA | |
| GCAGGCTCCAGCGCGGAGGGCCCGGAGTTTCCCGGCTCACGGGCAG | |
| CGTGTACCAGCCTCTCCAGCCCCGACGCCTTTGCGAGTTGGGTTTTT | |
| ATCCCGGAGCTGATGGGCTGTGCACGTGGACCTGTCTCTTCCCTCTG | |
| GGCTGCGGTTTTAGAGGTGGGGGACCCACGGCCCACGCTCCCTGGG | |
| CCTCTGATCCCCGCATCAGATTCCGAGAGGGCACCCTGGGCGCTGT | |
| CTCCCCCACTGCCAGCCAGCGTCTGCCTCCCTCCCAGGAGCCCTCAC | |
| ATGGCACCCGCTTCTCCCTGGATACTCTGGTGAGGGAAGCCCAAAC | |
| TCGCCATGATGCCCACAGCTTTTGTCTAGAAATCGGAATTTCGGGC | |
| GATGCTAGACTCCAGCGGGCCCTGGGCGTCAGAAGGAATCTCATTC | |
| TCGAACGAGCCGCACGGCCGTTCGCTTCCTCTTGGGTCCGGGACCT | |
| GGGCCCAGGCCTGTTTCCCCGTCTGCGCCGGGGGTAGTAGGACAGG | |
| GCCACCTGAGCAGAGGGCGGAGGCCCCAGCAGTGGTGCGTGGGCT | |
| CTGGTTCTGAGACCCTGGCCCAGAGCCGCTCGCTCACTGCAGTCGC | |
| CGTGCCTGTCGCTCCAGTACTGACCAAGTGCCAGGAAGAGGTCAGC | |
| CGTGTCCCCGCCGTCCACCCGGGCATGTTCAGGCCCAAGTGCGACG | |
| AGGACGGCAACTACATGCCGCTCCAGTGCTACGGGAGCATCGGCTT | |
| CTGCTGGTGCGTCTTCCCCAACGGCACCGAGGTCCCGCACACCAGG | |
| AGCCGCGGGCGCCACAGCTGCAGCGGTAAGCGGTGGCCTCGGCGC | |
| CAGGAGCAGGAGGGCCCAGGGCCAGGACGGCAAGGCGGCCACTCC | |
| TGGGGGCAGCCTCTGGCTGTACGCCCACCTGTCTGCCCCTCACATCC | |
| GCACTCCTGTCTGATCCGATCTGTACCCGTCAAAGCGCCGCCCGGC | |
| CACGCAGCCAGGCCTCCAGACCCGCGTCCTTCCTCACTCACTTCCCG | |
| GCCACACTGCACGGCCCCTGCCTGCCATCCGGCAAGCATCCCTGCT | |
| TGGCCAGGCGCCGGGCCTGCGGCGAGATGGGCTAGAACGAGCCCA | |
| CCCAGGCCCCTGCCAGCGGTGCCCCAGGCTGGCCTGCGCTGACAGG | |
| GGACCCTCTGCTCCTTTGCAGACCCACTGGAGACGGAGGACCTGTC | |
| GTCCGGGCTGGGCGTGACCAAGCAGGATCTCGGCCAAGGTAAGGG | |
| CCCTGCCTCCCAGCAGCCCAACCCCAGGGGGGCTGACGTCCCCCAG | |
| CACCCATCTGGCGGAGGAGGCCGGGGGCTGGGGGTCTCAGGGGCT | |
| CCGACAGGCAGGCCTTGTCCTCACGTAGGAGGGTGAGGCCACAGG | |
| GTGCTGGGGCGGGGGGAGCAGGGACAGTAGGGGAGCCCAACCCTA | |
| AGCCACTGCCCCTCTCTCCAGTCATCCTGTAAGACCAGCAGACGCC | |
| AGCCCCCAGCCCTGCGTGGCCACAGCTTGCCTGCCCTCCACCCTCCC | |
| TGCCGCCCCCACCTGCACTTCATCCCATGGGCCTCTGGCACCTGGCT | |
| CGCCGTCCTCCCTGGATACCTCACGTCTCTCAGAAGGCCAAGGAT | |
| GAACGACACAGGGAGGCCCTGCTGCCCACAGCTCCATCCGCTAGCA | |
| GGGACACAGGGCCCCGGGGACAGCCCGTGAGCGGGGCTGGTGCCA | |
| CTCCTGACACCTTGAGCTCCAACATGGCGGCTGTCCATGGATGAGG | |
| CGGGCAGGTGCCTGGGGACGGGTCCGCTCGATCCCAAACCCCCACA | |
| AGGACAGCTCTGCTGTCTCCTCCCCTGGTGGCCTCAGCTTCGCTGCC | |
| CGTTACCTGGGGAGACGGGCCAAGCCTTTGTGTGTCCCCAGCATCA | |
| GCCACCCACGGCCCCTCCTCACCCCCTGCCCGCCCCACATCCCTGTG | |
| CACAGTCAGGCTCGGCTCAGCCCCTGGCCACGGCTTCTGTGAGAAT | |
| AAAAGGTAGTGAATTAGGAC | |

Further provided are nucleotides at least 10, 15, 17, 20, 25, 30, 50, 100, 200, 250, 300, 350, 400, 450, 500, 550, 600, 650, 750, 850, 1000, 2775, 2800, 2900, 3000, 3100, 3200, 3300, 3400, 3500, 3600, 3700, 3800, 3900, 4000, 4500, 5000, 5500, 6000, 6500, 7000, 7500, 8000, or 8400 contiguous nucleotides of SEQ ID No. 19.

### III. Oligonucleotide Probes and Primers

The present invention further provides oligonucleotide probes and primers which hybridize to the hereinabove-described sequences (SEQ ID Nos. 1, 3-18, and 19). Oligonucleotides are provided that can be homologous to SEQ ID Nos. 1, 3-18, and 19, and fragments thereof. Oligonucleotides that hybridize under stringent conditions to SEQ ID Nos. 1, 3-18, and 19 and fragments thereof, are also provided. Stringent conditions can describe conditions under which hybridization will occur only if there is at least about 85%, about 90%, about 95%, or at least about 98% homology between the sequences. Alternatively, the oligonucleotide can have at least 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 75 or 100 bases which hybridize to SEQ ID Nos. 1, 3-18, and 19, and fragments thereof. Such oligonucleotides can be used as primers and probes to detect the sequences provided herein. The probe or primer can be at least 14 nucleotides in length, and in a preferred embodiment, are at least 15, 20, 25, 28, 30, or 35 nucleotides in length.

Given the above sequences, one of ordinary skill in the art using standard algorithms can construct oligonucleotide probes and primes that are complementary to sequences contained in Seq ID Nos. 1, 3-18, and 19, and fragments thereof. The rules for complementary pairing are well known: cytosine (“C”) always pairs with guanine (“G”) and thymine (“T”) or uracil (“U”) always pairs with adenine (“A”). It is recognized that it is not necessary for the primer or probe to be 100% complementary to the target nucleic acid sequence, as long as the primer or probe sufficiently hybridizes and can recognize the corresponding complementary sequence. A certain degree of pair mismatch can generally be tolerated.

Oligonucleotide sequences used as the hybridizing region of a primer can also be used as the hydridizing region of a probe. Suitability of a primer sequence for use as a probe depends on the hybridization characteristics of the primer. Similarly, an oligonucleotide used as a probe can be used as a primer.

It will be apparent to those skilled in the art that, provided with these specific embodiments, specific primers and probes can be prepared by, for example, the addition of nucleotides to either the 5′ or 3′ ends, which nucleotides are complementary to the target sequence or are not complimentary to the target sequence. So long as primer compositions serve as a point of initiation for extension on the target sequences, and so long as the primers and probes comprise at least 14 consecutive nucleotides contained within the above mentioned SEQ ID Nos. such compositions are within the scope of the invention.

The probes and primers herein can be selected by the following criteria, which are factors to be considered, but are not exclusive or determinative. The probes and primers are selected from the region of the porcine invariant chain nucleic acid sequence identified in SEQ ID Nos. 1, 3-18, 19, and fragments thereof. The probes and primers lack homology with sequences of other genes that would be expected to compromise the test. The probes or primers lack secondary structure formation in the amplified nucleic acid which can interfere with extension by the amplification enzyme such as E. coli DNA polymerase, preferably that portion of the DNA polymerase referred to as the Klenow fragment. This can be accomplished by employing up to about 15% by weight, preferably 5-10% by weight, dimethyl sulfoxide (DMSO) in the amplification medium and/or increasing the amplification temperatures to 300-40° C.

Preferably, the probes or primers should contain approximately 50% guanine and cytosine nucleotides, as measured by the formula adenine (A)+thymine (T)+cytosine (C)+guanine (G)/cytosine (C)+guanine (G). Preferably, the probe or primer does not contain multiple consecutive adenine and thymine residues at the 3′ end of the primer which can result in less stable hybrids.

The probes and primers of the invention can be about 10 to 30 nucleotides long, preferably at least 10, 11, 12, 13, 14, 15, 20, 25, or 28 nucleotides in length, including specifically 14, 15, 16, 17, 18, 19, 20, 21, 22, 23, 24, 25, 26, 27, 28, 29 or 30 nucleotides. The nucleotides as used in the present invention can be ribonucleotides, deoxyribonucleotides and modified nucleotides such as inosine or nucleotides containing modified groups which do not essentially alter their hybridization characteristics. Probe and primer sequences are represented throughout the specification as single stranded DNA oligonucleotides from the 5′ to the 3′ end. Any of the probes can be used as such, or in their complementary form, or in their RNA form (wherein T is replaced by U).

The probes and primers according to the invention can be prepared by cloning of recombinant plasmids containing inserts including the corresponding nucleotide sequences, optionally by cleaving the latter out from the cloned plasmids upon using the adequate nucleases and recovering them, e.g. by fractionation according to molecular weight. The probes and primers according to the present invention can also be synthesized chemically, for instance by the conventional phosphotriester or phosphodiester methods or automated embodiments thereof. In one such automated embodiment diethylphosphoramidites are used as starting materials and can be synthesized as described by Beaucage, et al., Tetrahedron Letters 22:1859-1862 (1981). One method of synthesizing oligonucleotides on a modified solid support is described in U.S. Pat. No. 4,458,066. It is also possible to use a probe or primer which has been isolated from a biological source (such as a restriction endonuclease digest).

The oligonucleotides used as primers or probes can also comprise nucleotide analogues such as phosphorothiates (Matsukura S., Naibunpi Gakkai Zasshi. 43(6):527-32 (1967)), alkylphosphorothiates (Miller P., et al., Biochemistry 18(23):5134-43 (1979), peptide nucleic acids (Nielsen P., et al., Science 254(5037):1497-500 (1991); Nielsen P., et al., Nucleic-Acids-Res. 21(2):197-200 (1993)), morpholino nucleic acids, locked nucleic acids, pseudocyclic oligonucleobases, 2′-O,4′-C-ethylene bridged nucleic acids or can contain intercalating agents (Asseline J., et al., Proc. Natl. Acad. Sci. USA 81(11):3297-301 (1984)).

For designing probes and primers with desired characteristics, the following useful guidelines known to the person skilled in the art can be applied. Because the extent and specificity of hybridization reactions are affected by a number of factors, manipulation of one or more of those factors will determine the exact sensitivity and specificity of a particular probe, whether perfectly complementary to its target or not. The importance and effect of various assay conditions, explained further herein, are known to those skilled in the art.

The stability of the probe and primer to target nucleic acid hybrid should be chosen to be compatible with the assay conditions. This can be accomplished by avoiding long AT-rich sequences, by terminating the hybrids with GC base pairs, and/or by designing the probe with an appropriate Tm. The beginning and end points of the probe should be chosen so that the length and % GC result in a Tm about 2-10° C. higher than the temperature at which the final assay will be performed. The base composition of the probe is significant because G-C base pairs exhibit greater thermal stability compared to A-T base pairs due to additional hydrogen bonding. Thus, hybridization involving complementary nucleic acids of higher G-C content will be stable at higher temperatures. Conditions such as ionic strength and incubation temperature under which probe will be used should also be taken into account when designing a probe. It is known that hybridization will increase as the ionic strength of the reaction mixture increases, and that the thermal stability of the hybrids will increase with increasing ionic strength. Chemical reagents, such as formamide, urea, DIVISO and alcohols, which disrupt hydrogen bonds, will increase the stringency of hybridization. Destabilization of the hydrogen bonds by such reagents can greatly reduce the Tm. In general, optimal hybridization for synthetic oligonucleotide probes of about 10-50 bases in length occurs approximately 5° C. below the melting temperature for a given duplex. Incubation at temperatures below the optimum can allow mismatched base sequences to hybridize and can therefore result in reduced specificity. It is desirable to have probes which hybridize only under conditions of high stringency. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. The degree of stringency is chosen such as to maximize the difference in stability between the hybrid formed with the target and the non-target nucleic acid. In the present case, single base pair changes need to be detected, which requires conditions of very high stringency.

The length of the target nucleic acid sequence and, accordingly, the length of the probe sequence can also be important. In some cases, there can be several sequences from a particular region, varying in location and length, which will yield probes and primers with the desired hybridization characteristics. In other cases, one sequence can be significantly better than another which differs merely by a single base.

While it is possible for nucleic acids that are not perfectly complementary to hybridize, the longest stretch of perfectly complementary base sequence will normally primarily determine hybrid stability. While oligonucleotide probes and primers of different lengths and base composition can be used, preferred oligonucleotide probes and primers of this invention are between about 14 and 30 bases in length and have a sufficient stretch in the sequence which is perfectly complementary to the target nucleic acid sequence.

Regions in the target DNA or RNA which are known to form strong internal structures inhibitory to hybridization are less preferred. Likewise, probes-with extensive self-complementarity should be avoided. As explained above, hybridization is the association of two single strands of complementary nucleic acids to form a hydrogen bonded double strand. It is implicit that if one of the two strands is wholly or partially involved in a hybrid, it will be less able to participate in formation of a new hybrid. There can be intramolecular and intermolecular hybrids formed within the molecules of one type of probe if there is sufficient self complementarity. Such structures can be avoided through careful probe design. By designing a probe so that a substantial portion of the sequence of interest is single stranded, the rate and extent of hybridization can be greatly increased. Computer programs are available to search for this type of interaction. However, in certain instances, it may not be possible to avoid this type of interaction.

Specific primers and sequence specific oligonucleotide probes can be used in a polymerase chain reaction that enables amplification and detection of porcine invariant chain nucleic acid sequences.

### IV. Genetic Targeting of the Porcine Invariant Chain Gene

Gene targeting allows for the selective manipulation of animal cell genomes. Using this technique, a particular DNA sequence can be targeted and modified in a site-specific and precise manner. Different types of DNA sequences can be targeted for modification, including regulatory regions, coding regions and regions of DNA between genes. Examples of regulatory regions include: promoter regions, enhancer regions, terminator regions and introns. By modifying these regulatory regions, the timing and level of expression of a gene can be altered. Coding regions can be modified to alter, enhance or eliminate the protein within a cell. Introns and exons, as well as inter-genic regions, are suitable targets for modification.

Modifications of DNA sequences can be of several types, including insertions, deletions, substitutions, or any combination thereof. A specific example of a modification is the inactivation of a gene by site-specific integration of a nucleotide sequence that disrupts expression of the gene product, i.e. a “knock out”. For example, one approach to disrupting the porcine invariant chain gene is to insert a selectable marker into the targeting DNA such that homologous recombination between the targeting DNA and the target DNA can result in insertion of the selectable marker into the coding region of the target gene. In this way, for example, the porcine invariant chain gene sequence is disrupted, rendering the encoded enzyme nonfunctional.

a. Homologous Recombination.

Homologous recombination permits site-specific modifications in endogenous genes and thus novel alterations can be engineered into the genome. A primary step in homologous recombination is DNA strand exchange, which involves a pairing of a DNA duplex with at least one DNA strand containing a complementary sequence to form an intermediate recombination structure containing heteroduplex DNA (see, Radding, C. M. (1982) Ann. Rev. Genet. 16: 405; U.S. Pat. No. 4,888,274). The heteroduplex DNA can take several forms, including a three DNA strand containing triplex form wherein a single complementary strand invades the DNA duplex (Hsieh et al. (1990) Genes and Development 4: 1951; Rao et al., (1991) PNAS 88:2984)) and, when two complementary DNA strands pair with a DNA duplex, a classical Holliday recombination joint or chi structure (Holliday, R. (1964) Genet. Res. 5: 282) can form, or a double-D loop (“Diagnostic Applications of Double-D Loop Formation” U.S. Ser. No. 07/755,462, filed Sep. 4, 1991, which is incorporated herein by reference). Once formed, a heteroduplex structure can be resolved by strand breakage and exchange, so that all or a portion of an invading DNA strand is spliced into a recipient DNA duplex, adding or replacing a segment of the recipient DNA duplex. Alternatively, a heteroduplex structure can result in gene conversion, wherein a sequence of an invading strand is transferred to a recipient DNA duplex by repair of mismatched bases using the invading strand as a template (Genes, 3rd Ed. (1987) Lewin, B., John Wiley, New York, N.Y.; Lopez et al. (1987) Nucleic Acids Res. 15: 5643). Whether by the mechanism of breakage and rejoining or by the mechanism(s) of gene conversion, formation of heteroduplex DNA at homologously paired joints can serve to transfer genetic sequence information from one DNA molecule to another.

The ability of homologous recombination (gene conversion and classical strand breakage/rejoining) to transfer genetic sequence information between DNA molecules makes targeted homologous recombination a powerful method in genetic engineering and gene manipulation.

In homologous recombination, the incoming DNA interacts with and integrates into a site in the genome that contains a substantially homologous DNA sequence. In non-homologous (“random” or “illicit”) integration, the incoming DNA is not found at a homologous sequence in the genome but integrates elsewhere, at one of a large number of potential locations. In general, studies with higher eukaryotic cells have revealed that the frequency of homologous recombination is far less than the frequency of random integration. The ratio of these frequencies has direct implications for “gene targeting” which depends on integration via homologous recombination (i.e. recombination between the exogenous “targeting DNA” and the corresponding “target DNA” in the genome).

A number of papers describe the use of homologous recombination in mammalian cells. Illustrative of these papers are Kucherlapati et al., Proc. Natl. Acad. Sci. USA 81:3153-3157, 1984; Kucherlapati et al., Mol. Cell. Bio. 5:714-720, 1985; Smithies et al, Nature 317:230-234, 1985; Wake et al., Mol. Cell. Bio. 8:2080-2089, 1985; Ayares et al., Genetics 111:375-388, 1985; Ayares et al., Mol. Cell. Bio. 7:1656-1662, 1986; Song et al., Proc. NatI. Acad. Sci. USA 84:6820-6824, 1987; Thomas et al. Cell 44:419-428, 1986; Thomas and Capecchi, Cell 51: 503-512, 1987; Nandi et al., Proc. Natl. Acad. Sci. USA 85:3845-3849, 1988; and Mansour et al., Nature 336:348-352, 1988. Evans and Kaufman, Nature 294:146-154, 1981; Doetschman et al., Nature 330:576-578, 1987; Thomas and Capecchi, Cell 51:503-512,4987; Thompson et al., Cell 56:316-321, 1989.

The present invention uses homologous recombination to inactivate the porcine invariant chain gene in cells, such as fibroblasts. The DNA can comprise at least a portion of the gene at the particular locus with introduction of an alteration into at least one, optionally both copies, of the native gene, so as to prevent expression of a functional invariant chain protein. The alteration can be an insertion, deletion, replacement or combination thereof. When the alteration is introduce into only one copy of the gene being inactivated, the cells having a single unmutated copy of the target gene are amplified and can be subjected to a second targeting step, where the alteration can be the same or different from the first alteration, usually different, and where a deletion, or replacement is involved, can be overlapping at least a portion of the alteration originally introduced. In this second targeting step, a targeting vector with the same arms of homology, but containing a different mammalian selectable marker can be used. The resulting transformants are screened for the absence of a functional target antigen and the DNA of the cell can be further screened to ensure the absence of a wild-type target gene. Alternatively, homozygosity as to a phenotype can be achieved by breeding hosts heterozygous for the mutation.

Porcine cells that can be genetically modified can be obtained from a variety of different organs and tissues such as, but not limited to, brain, heart, lungs, glands, brain, eye, stomach, spleen, pancreas, kidneys, liver, intestines, uterus, bladder, skin, hair, nails, ears, nose, mouth, lips, gums, teeth, tongue, salivary glands, tonsils, pharynx, esophagus, large intestine, small intestine, rectum, anus, pylorus, thyroid gland, thymus gland, suprarenal capsule, bones, cartilage, tendons, ligaments, skeletal muscles, smooth muscles, blood. vessels, blood, spinal cord, trachea, ureters, urethra, hypothalamus, pituitary, adrenal glands, ovaries, oviducts, uterus, vagina, mammary glands, testes, seminal vesicles, penis, lymph, lymph nodes and lymph vessels. In one embodiment of the invention, porcine cells can be selected from the group consisting of, but not limited to, epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, phosphate cells, cumulus cells, epidermal cells, endothelial cells, Islets of Langerhans cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, squamous epithelial cells, osteocytes, osteoblasts, and osteoclasts.

In one alternative embodiment, embryonic stem cells can be used. An embryonic stem cell line can be employed or embryonic stem cells can be obtained freshly from a host, such as a porcine animal. The cells can be grown on an appropriate fibroblast-feeder layer or grown in the presence of leukemia inhibiting factor (LIF). In a preferred embodiment, the porcine cells can be fibroblasts; in one specific embodiment, the porcine cells can be fetal fibroblasts. Fibroblast cells are a preferred somatic cell type because they can be obtained from developing fetuses and adult animals in large quantities.

These cells can be easily propagated in vitro with a rapid doubling time and can be clonally propagated for use in gene targeting procedures.

b. Targeting Vectors

Cells homozygous at a targeted locus can be produced by introducing DNA into the cells, where the DNA has homology to the target locus and includes a marker gene, allowing for selection of cells comprising the integrated construct. The homologous DNA in the target vector will recombine with the chromosomal DNA at the target locus. The marker gene can be flanked on both sides by homologous DNA sequences, a 3′recombination arm and a 5′recombination arm. Methods for the construction of targeting vectors have been described in the art, see, for example, Dai et al., Nature Biotechnology 20: 251-255, 2002; WO 00/51424.

Various constructs can be prepared for homologous recombination at a target locus. Usually, the construct can include at least 50 bp, 100 bp, 500 bp, 1 kbp, 2 kbp, 4 kbp, 5 kbp, 10 kbp, 15 kbp, 20 kbp, or 50 kbp of sequence homologous with the target locus. The sequence can include any contiguous sequence of the porcine invariant chain gene, including at least 5, 10, 15, 17, 20, 25, 26, 27, 28, 29, 30, 35, 40, 45, 50, 55, 60, 65, 70, 75, 80, 85, 90 95, 100, 110, 120, 130, 140, 150, 160, 170, 180, 190, 200, 225, 250, 275, 300, 325, 350, 375, 400, 425, 450, 475, 500, 550, 600, 700, 650, 750, 800, 850, 900, 1000 contiguous nucleotides of Seq ID Nos 3-18, 19 or Seq ID No 20 (Table 5) or any combination or fragment thereof. Fragments of Seq ID Nos. 3-18, 19, or Seq ID 20 (Table 5) can include any contiguous nucleic acid or peptide sequence that includes at least about 10 bp, 15 bp, 17 bp, 20 bp, 50 bp, 100 bp, 500 bp, 1 kbp, 5 kbp or 10 kpb. The construct can include a sequence which encodes a polypeptide comprising the amino acid sequence of Seq ID No. 2 or a nucleotide sequence encoding a polypeptide comprising an amino acid sequence which is homologous to Seq ID No. 2. The construct can also include a nucleotide sequence encoding a polypeptide comprising an amino acid sequence homologous to Seq ID No. 2 having at least 99%, at least 95%, at least 90%, at least 85%, at least 80%, at least 70%, at least 60%, at least 50%, at least 40% or at least 25% amino acid identity or similarity to a polypeptide comprising the sequence of Seq ID No. 2 or a nucleotide sequence encoding an amino acid sequence having at least 99%, at least 95%, at least 90%, at least 85%, at least 80%, at least 70%, at least 60%, at least 50%, at least 40% or at least 25% amino acid identity or similarity to a fragment comprising at least 5, 10, 15, 20, 25, 30, 35, 40, 50, 75, 100, 150, 200, 250, 300 or 350 consecutive amino acids of Seq ID No. 2. The percentage of similarity or identity to Seq ID No. 2 can be determined using the FASTA version 3.0t78 algorithm with the default parameters. Alternatively, the percentage of identity or similarity to Seq ID No. 2 can be determined using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety

TABLE 5
PORCINE INVARIANT CHAIN SEQ ID No 20

| | |
|---|---|
| GCTGGACAAGCTGACGGTCACCTCTCAGAACTTGC | Seq ID No 20 |
| AGCTGGAGAGCCTGCGGATGAAGCTTCCCAAGCC | |
| TCCAAGCCTTTGAGCAAGATGCGGGTTTCCGC | |

Various considerations can be involved in determining the extent of homology of target DNA sequences, such as, for example, the size of the target locus, availability of sequences, relative efficiency of double cross-over events at the target locus and the similarity of the target sequence with other sequences.

The targeting DNA can include a sequence in which DNA substantially isogenic flanks the desired sequence modifications with a corresponding target sequence in the genome to be modified. The substantially isogenic sequence can be at least about 95%, 97-98%, 99.0-99.5%, 99.6-99.9%, or 100% identical to the corresponding target sequence (except for the desired sequence modifications). The targeting DNA and the target DNA preferably can share stretches of DNA at least about 75, 150 or 500 base pairs that are 100% identical. Accordingly, targeting DNA can be derived from cells closely related to the cell line being targeted; or the targeting DNA can be derived from cells of the same cell line or animal as the cells being targeted.

The DNA constructs can be designed to modify the endogenous, target porcine invariant chain gene. The homologous sequence for targeting the construct can have one or more deletions, insertions, substitutions or combinations thereof. The alteration can be the insertion of a selectable marker gene fused in reading frame with the upstream sequence of the target gene.

Suitable selectable marker genes include, but are not limited to: genes conferring the ability to grow on certain media substrates, such as the tk gene (thymidine kinase) or the hprt gene (hypoxanthine phosphoribosyltransferase) which confer the ability to grow on HAT medium (hypoxanthine, aminopterin and thymidine); the bacterial gpt gene (guanine/xanthine phosphoribosyltransferase) which allows growth on MAX medium (mycophenolic acid, adenine, and xanthine). See Song et al., Proc. Nat'l Acad. Sci. U.S.A. 84:6820-6824 (1987). See also Sambrook et al., Molecular Cloning—A Laboratory Manual, Cold Spring Harbor Laboratory, Cold Spring Harbor, N.Y. (1989), see chapter 16. Other examples of selectable markers include: genes conferring resistance to compounds such as antibiotics, genes conferring the ability to grow on selected substrates, genes encoding proteins that produce detectable signals such as luminescence, such as green fluorescent protein, enhanced green fluorescent protein (eGFP). A wide variety of such markers are known and available, including, for example, antibiotic resistance genes such as the neomycin resistance gene (neo), Southern, P., and P. Berg, J. Mol. Appl. Genet. 1:327-341 (1982); and the hygromycin resistance gene (hyg), Nucleic Acids Research 11:6895-6911 (1983), and Te Riele et al., Nature 348:649-651 (1990). Other selectable marker genes include: acetohydroxy acid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline.

Methods for the incorporation of antibiotic resistance genes and negative selection factors will be familiar to those of ordinary skill in the art (see, e.g., WO 99/15650; U.S. Pat. No. 6,080,576; U.S. Pat. No. 6.136,566; Niwa, et al., J. Biochem. 113:343-349 (1993); and Yoshida, et al., Transgenic Research, 4:277-287 (1995)).

Additional selectable marker genes useful in this invention, for example, are described in U.S. Pat. Nos: 6,319,669; 6,316,181; 6,303,373; 6,291,177; 6,284,519; 6,284,496; 6,280,934; 6,274,354; 6,270,958; 6,268,201; 6,265,548; 6,261,760; 6,255,558; 6,255,071; 6,251,677; 6,251,602; 6,251,582; 6,251,384; 6,248,558; 6,248,550; 6,248,543; 6,232,107; 6,228,639; 6,225,082; 6,221,612; 6,218,185; 6,214,567; 6,214,563; 6,210,922; 6,210,910; 6,203,986; 6,197,928; 6,180,343; 6,172,188; 6,153,409; 6,150,176; 6,146,826; 6,140,132; 6,136,539; 6,136,538; 6,133,429; 6,130,313; 6,124,128; 6,110,711; 6,096,865; 6,096,717; 6,093,808; 6,090,919; 6,083,690; 6,077,707; 6,066,476; 6,060,247; 6,054,321; 6,037,133; 6,027,881; 6,025,192; 6,020,192; 6,013,447; 6,001,557; 5,994,077; 5,994,071; 5,993,778; 5,989,808; 5,985,577; 5,968,773; 5,968,738; 5,958,713; 5,952,236; 5,948,889; 5,948,681; 5,942,387; 5,932,435; 5,922,576; 5,919,445; and 5,914,233.

Combinations of selectable markers can also be used. For example, to target porcine invariant chain gene, a neo gene (with or without its own promoter, as discussed above) can be cloned into a DNA sequence which is homologous to the porcine invariant chain gene. To use a combination of markers, the HSV-tk gene can be cloned such that it is outside of the targeting DNA (another selectable marker could be placed on the opposite flank, if desired). After introducing the DNA construct into the cells to be targeted, the cells can be selected on the appropriate antibiotics. In this particular example, those cells which are resistant to G418 and gancyclovir are most likely to have arisen by homologous recombination in which the neo gene has been recombined into the porcine invariant chain gene but the tk gene has been lost because it was located outside the region of the double crossover.

Deletions can be at least about 50 bp, more usually at least about 100 bp, and generally not more than about 20 kbp, where the deletion can normally include at least a portion of the coding region including a portion of or one or more exons, a portion of or one or more introns, and can or can not include a portion of the flanking non-coding regions, particularly the 5′-non-coding region (transcriptional regulatory region). Thus, the homologous region can extend beyond the coding region into the 5′-non-coding region or alternatively into the 3′-non-coding region. Insertions can generally not exceed 10 kbp, usually not exceed 5 kbp, generally being at least 50 bp, more usually at least 200 bp.

The region(s) of homology can include mutations, where mutations can further inactivate the target gene, in providing for a frame shift, or changing a key amino acid, or the mutation can correct a dysfunctional allele, etc. Usually, the mutation can be a subtle change, not exceeding about 5% of the homologous flanking sequences. Where mutation of a gene is desired, the marker gene can be inserted into an intron, so as to be excised from the target gene upon transcription.

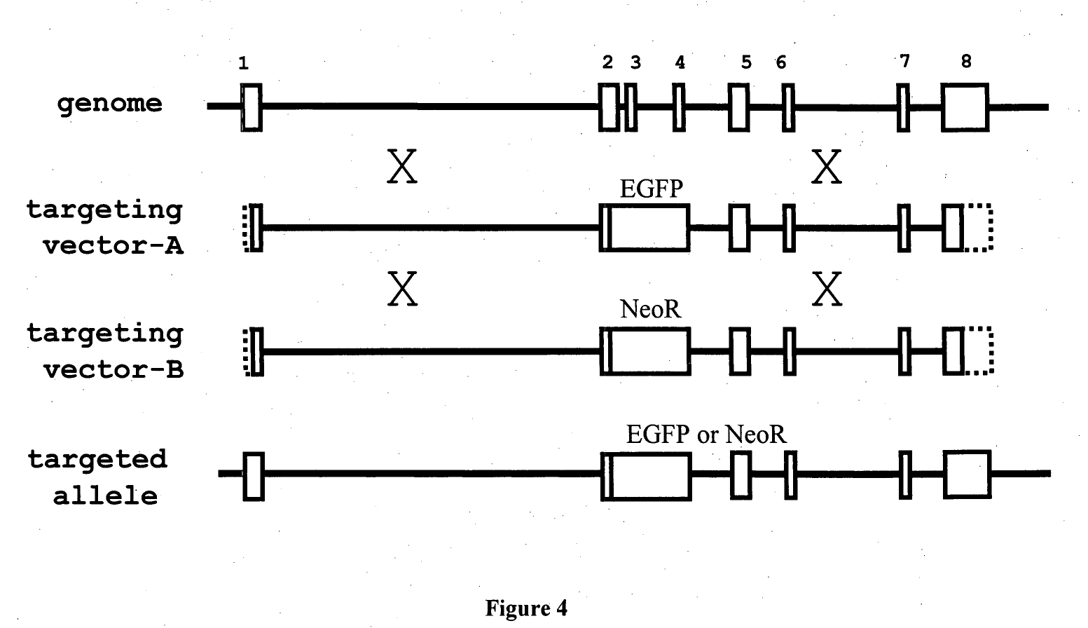

The construct can be prepared in accordance with methods known in the art, various fragments can be brought together, introduced into appropriate vectors, cloned, analyzed and then manipulated further until the desired construct has been achieved see, for example, FIG. 4). Various modifications can be made to the sequence, to allow for restriction analysis, excision, identification of probes, etc. Silent mutations can be introduced, as desired. At various stages, restriction analysis, sequencing, amplification with the polymerase chain reaction, primer repair, in vitro mutagenesis, etc. can be employed.

The construct can be prepared using a bacterial vector, including a prokaryotic replication system, e.g. an origin recognizable by E. coli, at each stage the construct can be cloned and analyzed. A marker, the same as or different from the marker to be used for insertion, can be employed, which can be removed prior to introduction into the target cell. Once the vector containing the construct has been completed, it can be further manipulated, such as by deletion of the bacterial sequences, linearization, introducing a short deletion in the homologous sequence. After final manipulation, the construct can be introduced into the cell.

Techniques which can be used to allow the DNA construct entry into the host cell include calcium phosphate/DNA coprecipitation, microinjection of DNA into the nucleus, electroporation, bacterial protoplast fusion with intact cells, transfection, or any other technique known by one skilled in the art. The DNA can be single or double stranded, linear or circular, relaxed or supercoiled DNA. For various techniques for transfecting mammalian cells, see, for example, Keown et al., Methods in Enzymology Vol. 185, pp. 527-537 (1990).

The present invention further includes recombinant constructs comprising one or more of the sequences as broadly described above (for example in Tables 3-5). The constructs comprise a vector, such as a plasmid or viral vector, into which a sequence of the invention has been inserted, in a forward or reverse orientation. The construct can also include regulatory sequences, including, for example, a promoter, operably linked to the sequence. Large numbers of suitable vectors and promoters are known to those of skill in the art, and are commercially available. The following vectors are provided by way of example: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSv2cat, pOG44, pXT1, pSG (Stratagene) pSVK3, pBPv, pMSG, pSVL (Pharmiacia). Also, any other plasmids and vectors can be used as long as they are replicable and viable in the host. Vectors known in the art and those commercially available (and variants or derivatives thereof) can in accordance with the invention be engineered to include one or more recombination sites for use in the methods of the invention. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, -pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMClneo, and pOG44 (Stratagene, Inc.), and pYES2, pAC360, pBlueBacHis A, B, and C, pVL1392, pBlueBacIII, pCDM8, pcDNA1, pZeoSV, pcDNA3 pREP4, pCEP4, and pEBVHis (Invitrogen, Corp.) and variants or derivatives thereof.

Other vectors suitable for use in the invention include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen) and variants or derivatives thereof. Viral vectors can also be used, such as lentiviral vectors (see, for example, WO 03/059923; Tiscornia et al. PNAS 100:1844-1848 (2003)).

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pPICZA, pPICZB, pPICZC, pGAPZA, pGAPZB, pGAPZC, pBlueBac4.5, pBlueBacHis2, pMelBac, pSinRep5, pSinHis, pIND, pIND(SP1), pVgRXR, pcDNA2.1, pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; λ ExCell, λ gt11, pTrc99A, pKK223-3, pGEX-1 λ T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32LIC, pET-30LIC, pBAC-2cp LIC, pBACgus-2cp LIC, pT7Blue-2 LIC, pT7Blue-2, λ SCREEN-1, λ BlueSTAR, pET-3abcd, pET-7abc, pET9abcd, pET11abcd, pET12abc, pET-14b, pET-lSb, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3cp, pBACgus-2cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, pβgal-Enhancer, pCMV, pTet-Off, pTet-On, pTK-Hyg, pRetro-Off, pRetro-On, pIRESlneo, pIRESlhyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTrip1Ex, λgt10, λgt11, pWE15, and λTrip1Ex from Clontech; Lambda ZAP II, pBK-CMV, pBK-RSV, pBluescript II KS ±, pBluescript II SK ±, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS ±, pBC KS ±, pBC SK ±, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOP13 CAT,pXT1, pSG5, pPbac, pMbac, pMC1neo, pMC1neo Poly A, pOG44, pOG45, pFRTβGAL, pNEOβGAL, pRS403, pRS404, pRS405, pRS406, pRS413, pRS414, pRS415, and pRS416 from Stratagene.

Additional vectors include, for example, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Also, any other plasmids and vectors known in the art can be used as long as they are replicable and viable in the host.

c. Selection of Homologously Recombined Cells

Cells that have been homologously recombined to knock-out expression of the porcine invariant chain gene can then be grown in appropriately-selected medium to identify cells providing the appropriate integration. Those cells which show the desired phenotype can then be further analyzed by restriction analysis, electrophoresis, Southern analysis, polymerase chain reaction, or another technique known in the art. By identifying fragments which show the appropriate insertion at the target gene site, cells can be identified in which homologous recombination has occurred to inactivate or otherwise modify the target gene.

The presence of the selectable marker gene inserted into the porcine invariant chain gene establishes the integration of the target construct into the host genome. Those cells which show the desired phenotype can then be further analyzed by restriction analysis, electrophoresis, Southern analysis, polymerase chain reaction, etc to analyze the DNA in order to establish whether homologous or non-homologous recombination occurred. This can be determined by employing probes for the insert and then sequencing the 5′ and 3′ regions flanking the insert for the presence of the invariant chain gene extending beyond the flanking regions of the construct or identifying the presence of a deletion, when such deletion is introduced. Primers can also be used which are complementary to a sequence within the construct and complementary to a sequence outside the construct and at the target locus. In this way, one can only obtain DNA duplexes having both of the primers present in the complementary chains if homologous recombination has occurred. By demonstrating the presence of the primer sequences or the expected size sequence, the occurrence of homologous recombination is supported.

The polymerase chain reaction used for screening homologous recombination events is described in Kim and Smithies, Nucleic Acids Res. 16:8887-8903, 1988; and Joyner et al., Nature 338:153-156, 1989. The combination of a mutant polyoma enhancer and a thymidine kinase promoter to drive the neomycin gene has been shown to be active in both embryonic stem cells and EC cells by Thomas and Capecchi, supra, 1987; Nicholas and Berg (1983) in Teratocarcinoma Stem Cell, eds. Siver, Martin and Strikland (Cold Spring Harbor Lab., Cold Spring Harbor, N.Y. (pp. 469-497); and Linney and Donerly, Cell 35:693-699, (1983).

An alternative method for screening homologous recombination events includes utilizing monoclonal or polyclonal antibodies specific for porcine invariant chain protein.

Further characterization of porcine cells lacking expression of functional porcine invariant chain due to homologous recombination events include, but are not limited to, Southern Blot analysis, Northern Blot analysis, and/or sequence analysis, or by using anti-invariant chain antibody assays.

The cell lines obtained from the first round of targeting are likely to be heterozygous for the targeted allele. Homozygosity, in which both alleles are modified, can be achieved in a number of ways. One approach is to grow up a number of cells in which one copy has been modified and then to subject these cells to another round of targeting using a different selectable marker. Alternatively, homozygotes can be obtained by breeding animals heterozygous for the modified allele, according to traditional Mendelian genetics. In some situations, it can be desirable to have two different modified alleles. This can be achieved by successive rounds of gene targeting or by breeding heterozygotes, each of which carries one of the desired modified alleles.

### V. Genetic Manipulation of Additional Genes to Overcome Immunolagic Barriers of Xenotransplatiation

In one aspect of the invention, cells homozygous for the nonfunctional porcine invariant chain gene can be subject to further genetic modification. For example, one can introduce additional genetic capability into the homozygotic hosts, where the endogenous alleles have been made nonfunctional, to substitute, replace or provide different genetic capability to the host. One can remove the marker gene after homogenotization. By introducing a construct comprising substantially the same homologous DNA, possibly with extended sequences, having the marker gene portion of the original construct deleted, one can be able to obtain homologous recombination with the target locus. By using a combination of marker genes for integration, one providing positive selection and the other negative selection, in the removal step, one would select against the cells retaining the marker genes.

In one embodiment, Porcine cells are provided that lack the porcine invariant chain gene and the α1,3galactosyltransferase gene. Animals lacking functional porcine invariant chain gene can be produced according to the present invention, and then cells from this animal can be used to knockout the α1,3galactosyltransferase gene. Heterozygous and homozygous α1,3galactosyltransferase-negative porcine have recently been reported (see, for example, Phelps, et al., Science, 299: pp. 411-414 (2003)), WO 2004/028243, Dai et al. Science 2003) Alternatively, cells from these α1,3galactosyltransferase knockout animals can be used and further modified to inactivate the porcine invariant chain gene.

In another embodiment, porcine cells are provided that lack the porcine invariant chain gene and produce human complement inhibiting proteins. Animals lacking functional porcine invariant chain gene can be produced according to the present invention, and then cells from this animal can be further modified to express complement inhibiting proteins, such as human pr porcine complement inhibiting proteins, inclusing, but not limited to, CD59 (cDNA reported by Philbrick, W. M., et al. Eur. J. Immunol. 20:87-92 (1990)),human decay accelerating factor (DAF)(cDNA reported by Medof et al., Proc. NatI. Acad. Sci. USA 84: 2007 (1987)), and human membrane cofactor protein (MCP) (cDNA reported by Lublin, D. et al., J. Exp. Med. 168: 181-194, (1988)).

Transgenic pigs producing human complement inhibiting proteins are known in the art (see, for example, U.S. Pat. No. 6,166,288). Alternatively, cells from these transgenic pigs producing human complement inhibiting proteins can be used and further modified to inactivate the porcine invariant chain gene.

### VI. Production of Genetically Modified Animals

The present invention provides methods of producing a transgenic pig that lacks, expression of porcine invariant chain through the genetic modification of porcine totipotent embryonic cells. In one embodiment, the animals can be produced by: (a) identifying one or more target porcine invariant chain nucleic acid genomic sequences in an animal; (b) preparing one or more homologous recombination vectors targeting the porcine invariant chain nucleic acid genomic sequences; (c) inserting the one or more targeting vectors into the genomes of a plurality of totipotent cells of the animal, thereby producing a plurality of transgenic totipotent cells; (d) obtaining a tetraploid blastocyst of the animal; (e) inserting the plurality of totipotent cells into the tetraploid blastocyst, thereby producing a transgenic embryo; (f) transferring the embryo to a recipient female animal; and (g) allowing the embryo to develop to term in the female animal. The method of transgenic animal production described here by which to generate a transgenic pig is further generally described in U.S. Pat. No. 6,492,575.

In another embodiment, the totipotent cells can be embryonic stem (ES) cells. The isolation of ES cells from blastocysts, the establishing of ES cell lines and their subsequent cultivation are carried out by conventional methods as described, for example, by Doetchmann et al., J. Embryol. Exp. Morph. 87:27-45 (1985); Li et al., Cell 69:915-926 (1992); Robertson, E. J. “Tetracarcinomas and Embryonic Stem Cells: A Practical Approach,” ed. E. J. Robertson, IRL Press, Oxford, England (1987); Wurst and Joyner, “Gene Targeting: A Practical Approach,” ed. A. L. Joyner, IRL Press, Oxford, England (1993); Hogen et al., “Manipulating the Mouse Embryo: A Laboratory Manual,” eds. Hogan, Beddington, Costantini and Lacy, Cold Spring Harbor Laboratory Press, New York (1994); and Wang, et al., Nature 336:741-744 (1992). For example, after transforming embryonic stem cells with the targeting vector to alter the porcine invariant chain gene, the cells can be plated onto a feeder layer in an appropriate medium, for example, such as fetal bovine serum enhanced DMEM. Cells containing the construct can be detected by employing a selective medium, and after sufficient time for colonies to grow, colonies can be picked and analyzed for the occurrence of homologous recombination. Polymerase chain reaction can be used, with primers within and without the construct sequence but at the target locus. Those colonies which show homologous recombination can then be used for embryo manipulating and blastocyst injection. Blastocysts can be obtained from superovulated females. The embryonic stem cells can then be trypsinized and the modified cells added to a droplet containing the blastocysts. At least one of the modified embryonic stem cells can be injected into the blastocoel of the blastocyst. After injection, at least one of the blastocysts can be returned to each uterine horn of pseudopregnant females. Females are then allowed to go to term and the resulting litters screened for mutant cells having the construct. The blastocysts are selected for different parentage from the transformed ES cells. By providing for a different phenotype of the blastocyst and the ES cells, chimeric progeny can be readily detected, and then genotyping can be conducted to probe for the presence of the modified porcine invariant chain gene.

In a further embodiment of the invention, the totipotent cells can be embryonic germ (EG) cells. Embryonic Germ cells are undifferentiated cells functionally equivalent to ES cells, that is they can be cultured and transfected in vitro, then contribute to somatic and germ cell lineages of a chimera (Stewart et al., Dev. Biol. 161:626-628 (1994)). EG cells are derived by culture of primordial germ cells, the progenitors of the gametes, with a combination of growth factors: leukemia inhibitory factor, steel factor and basic fibroblast growth factor (Matsui, et al., Cell 70:841-847 (1992); Resnick, et al., Nature 359:550-551 (1992)). The cultivation of EG cells can be carried out using methods known to one skilled in the art, such as described in Donovan et al., “Transgenic Animals, Generation and Use,” Ed. L. M. Houdebine, Harwood Academic Publishers (1997).

Tetraploid blastocysts for use in the invention can be obtained by natural zygote production and development, or by known methods by electrofusion of two-cell embryos and subsequently cultured as described, for example, by James, et al., Genet. Res. Camb. 60:185-194 (1992); Nagy and Rossant, “Gene Targeting: A Practical Approach,” ed. A. L. Joyner, IRL Press, Oxford, England (1993); or by Kubiak and Tarkowski, Exp. Cell Res. 157:561-566 (1985).

The introduction of the ES cells or EG cells into the blastocysts can be carried out by any method known in the art, for example, as described by Wang, et al., EMBO J. 10:2437-2450 (1991).

A “plurality” of totipotent cells can encompass any number of cells greater than one. For example, the number of totipotent cells for use in the present invention can be about 2 to about 30 cells, about 5 to about 20 cells, or about 5 to about 10 cells. In one embodiment, about 5-10 ES cells taken from a single cell suspension are injected into a blastocyst immobilized by a holding pipette in a micromanipulation apparatus. Then the embryos are incubated for at least 3 hours, possibly overnight, prior to introduction into a female recipient animal via methods known in the art (see for example Robertson, E. J. “Teratocarcinomas and Embryonic Stem Cells: A Practical Approach” IRL Press, Oxford, England (1987)). The embryo can then be allowed to develop to term in the female animal.

### Somatic Cell Nuclear Tramsfer tp Produce Cloned, Transgenic Offspring

The present invention provides a method for cloning a pig lacking a functional porcine invariant chain gene via somatic cell nuclear transfer. In general, a wide variety of methods to accomplish mammalian cloning are currently being rapily developed and reported, any method that accomplishes the desired result can be used in the present invention. Nonlimiting examples of such methods are described below. For example, the pig can be produced by a nuclear transfer process comprising the following steps: obtaining desired differentiated pig cells to be used as a source of donor nuclei; obtaining oocytes from a pig; enucleating the oocytes; transferring the desired differentiated cell or cell nucleus into the enucleated oocyte, e.g., by fusion or injection, to form NT units; activating the resultant NT unit; and transferring said cultured NT unit to a host pig such that the NT unit develops into a fetus.

Nuclear transfer techniques or nuclear transplantation techniques are known in the art(Campbell et al, Theriogenology, 43:181 (1995); Collas, et al, Mol. Report Dev., 38:264-267 (1994); Keefer et al, Biol. Reprod., 50:935-939 (1994); Sims, et al, Proc. Natl. Acad. Sci., USA, 90:6143-6147 (1993); WO 94/26884; WO 94/24274, and WO 90/03432, U.S. Pat. Nos. 4,944,384 and 5,057,420). In one nonlimiting example, methods are provided such as those described in U.S. patent Publication Ser. No. 2003/0046722 to Collas, et al., which describes methods for cloning mammals that allow the donor chromosomes or donor cells to be reprogrammed prior to insertion into an enucleated oocyte. The invention also describes methods of inserting or fusing chromosomes, nuclei or cells with oocytes.

A donor cell nucleus, which has been modified to alter the invariant chain gene, is transferred to a recipient porcine oocyte. The use of this method is not restricted to a particular donor cell type. The donor cell can be as described in Wilmut, et al., Nature 385 810 (1997); Campbell, et al., Nature 380 64-66 (1996); or Cibelli, et al., Science 280 1256-1258 (1998). All cells of normal karyotype, including embryonic, fetal and adult somatic cells which can be used successfully in nuclear transfer can in principle be employed. Fetal fibroblasts are a particularly useful class of donor cells. Generally suitable methods of nuclear transfer are described in Campbell, et al., Theriogenology 43 181 (1995), Collas, et al., Mol. Reprod. Dev. 38 264-267 (1994), Keefer, et al., Biol. Reprod. 50 935-939 (1994), Sims, et al., Proc. Nat'l. Acad. Sci. USA 90 6143-6147 (1993), WO-A-9426884, WO-A-9424274, WO-A-9807841, WO-A-9003432, U.S. Pat. No. 4,994,384 and U.S. Pat. No. 5,057,420. Differentiated or at least partially differentiated donor cells can also be used. Donor cells can also be, but do not have to be, in culture and can be quiescent. Nuclear donor cells which are quiescent are cells which can be induced to enter quiescence or exist in a quiescent state in vivo. Prior art methods have also used embryonic cell types in cloning procedures (Campbell, et al. (Nature, 380:64-68, 1996) and Stice, et al (Biol. Reprod., 20 54:100-110, 1996).

Somatic nuclear donor cells may be obtained from a variety of different organs and tissues such as, but not limited to, skin, mesenchyme, lung, pancreas, heart, intestine, stomach, bladder, blood vessels, kidney, urethra, reproductive organs, and a disaggregated preparation of a whole or part of an embryo, fetus or adult animal. In a suitable embodiment of the invention, nuclear donor cells are selected from the group consisting of epithelial cells, fibroblast cells, neural cells, keratinocytes, hematopoietic cells, melanocytes, chondrocytes, lymphocytes (B and T), macrophages, monocytes, mononuclear cells, cadiac muscle cells, other muscle cells, granulose cells, cumulus cells, epidermal cells or endothelial cells. In another embodiment, the nuclear cell is an embryonic stem cell. In a preferred embodiment, fibroblast cells can be used as donor cells.

In another embodiment of the invention, the nuclear donor cells of the invention are germ cells of an animal. Any germ cell of an animal species in the embryonic, fetal, or adult stage may be used as a nuclear donor cell. In a suitable embodiment, the nuclear donor cell is an embryonic germ cell.

Nuclear donor cells may be arrested in any phase of the cell cycle (GO, GI, G2, S, M) so as to ensure coordination with the acceptor cell. Any method known in the art may be used to manipulate the cell cycle phase. Methods to control the cell cycle phase include, but are not limited to, GO quiescence induced by contact inhibition of cultured cells, GO quiescence induced by removal of serum or other essential nutrient, GO quiescence induced by senescence, GO quiescence induced by addition of a specific growth factor; GO or GI quiescence induced by physical or chemical means such as heat shock, hyperbaric pressure or other treatment with a chemical, hormone, growth factor or other substance; S-phase control via treatment with a chemical agent which interferes with any. Point of the replication procedure; M-phase control via selection using fluorescence activated cell sorting, mitotic shake. off, treatment with microtubule disrupting agents or any chemical which disrupts progression in mitosis (see also Freshney, R. I,. “Culture of.Animal Cells: A Manual of Basic Technique,” Alan R. Liss, Inc, New York (1983).

Methods for isolation of oocytes are well known in the. art. Essentially, this can comprise isolating oocytes from the ovaries or reproductive tract of a pig. A readily available source of pig oocytes is slaughterhouse materials. For the combination of techniques such as genetic engineering, nuclear transfer and cloning, oocytes must generally be matured in vitro before these cells can be used as recipient cells for nuclear transfer, and before they can be fertilized by the sperm cell to develop into an embryo. This process generally requires collecting immature (prophase I) oocytes from mammalian ovaries, e.g., bovine ovaries obtained at a slaughterhouse, and maturing the oocytes in a maturation medium prior to fertilization or enucleation until the oocyte attains the metaphase II stage, which in the case of bovine oocytes generally occurs about 18-24 hours post-aspiration. This period of time is known as the “maturation period”.

A metaphase II stage oocyte can be the recipient oocyte, at this stage it is believed that the oocyte can be or is sufficiently “activated” to treat the introduced nucleus as it does a fertilizing sperm. Metaphase II stage oocytes, which have been matured in vivo have been successfully used in nuclear transfer techniques. Essentially, mature metaphase II oocytes can be collected surgically from either non-superovulated or superovulated porcine 35 to 48, or 39-41, hours past the onset of estrus or past the injection of human chorionic gonadotropin (hCG) or similar hormone.

After a fixed time maturation period, which ranges from about 10 to 40 hours, and preferably about 16-18 hours, the oocytes can be enucleated. Prior to enucleation the oocytes can be removed and placed in appropriate medium, such as HECM containing 1 milligram per milliliter of hyaluronidase prior to removal of cumulus cells. The stripped oocytes can then be screened for polar bodies, and the selected metaphase II oocytes, as determined by the presence of polar bodies, are then used for nuclear transfer. Enucleation follows.

Enucleation can be performed by known methods, such as described in U.S. Pat. No. 4,994,384. For example, metaphase II oocytes can be placed in either HECM, optionally containing 7.5 micrograms per milliliter cytochalasin B, for immediate enucleation, or can be placed in a suitable medium, for example an embryo culture medium such as CR1aa, plus 10% estrus cow serum, and then enucleated later, preferably not more than 24 hours later, and more preferably 16-18 hours later. Enucleation can be accomplished microsurgically using a micropipette to remove the polar body and the adjacent cytoplasm. The oocytes can then be screened to identify those of which have been successfully enucleated. One way to screen the oocytes is to stain the oocytes with 1 microgram per milliliter 33342 Hoechst dye in HECM, and then view the oocytes under ultraviolet irradiation for less than 10 seconds. The oocytes that have been successfully enucleated can then be placed in a suitable culture medium, for example, CR1aa plus 10% serum.

A single mammalian cell of the same species as the enucleated oocyte can then be transferred into the perivitelline space of the enucleated oocyte used to produce the NT unit. The mammalian cell and the enucleated oocyte can be used to produce NT units according to methods known in the art. For example, the cells can be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels can open between the two cells. Due to the thermodynamic instability of such a small opening, it enlarges until the two cells become one. See, for example, U.S. Pat. No. 4,997,384 by Prather et al. A variety of electrofusion media can be used including, for example, sucrose, mannitol, sorbitol and phosphate buffered solution. Fusion can also be accomplished using Sendai virus as a fusogenic agent (Graham, Wister Inot. Symp. Monogr., 9, 19, 1969). Also, the nucleus can be injected directly into the oocyte rather than using electroporation fusion. See, for example, Collas and Barnes, Mol. Reprod Dev., 38:264-267 (1994). After fusion, the resultant fused NT units are then placed in a suitable medium until activation, for example, CR1aa medium. Typically activation can be effected shortly thereafter, for example less than 24 hours later, or about 4-9 hours later.

The NT unit can be activated by any method that accomplishes the desired result. Such methods include, for example, culturing the NT unit at sub-physiological temperature, in essence by applying a cold, or actually cool temperature shock to the NT unit. This can be most conveniently done by culturing the NT unit at room temperature, which is cold relative to the physiological temperature conditions to which embryos are normally exposed. Alternatively, activation can be achieved by application of known activation agents. For example, penetration of oocytes by sperm during fertilization has been shown to activate prefusion oocytes to yield greater numbers of viable pregnancies and multiple genetically identical pigs after nuclear transfer. Also, treatments such as electrical and chemical shock can be used to activate NT embryos after fusion. See, for example, U.S. Pat. No. 5,496,720, to Susko-Parrish, et al. Additionally, activation can be effected by simultaneously or sequentially by increasing levels of divalent cations in the oocyte, and reducing phosphorylation of cellular proteins in the oocyte. This can generally be effected by introducing divalent cations into the oocyte cytoplasm, e.g.,. magnesium, strontium, barium or calcium, e.g., in the form of an ionophore. Other methods of increasing divalent cation levels include the use of electric shock, treatment with ethanol and treatment with caged chelators. Phosphorylation can be reduced by known methods, for example, by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins can be inhibited by introduction of a phosphatase into the oocyte, e.g., phosphatase 2A and phosphatase 2B.

The activated NT units can then be cultured in a suitable in vitro culture medium until the generation of cell colonies. Culture media suitable for culturing and maturation of embryos are well known in the art. Examples of known media, which can be used for embryo culture and maintenance, include Ham's F−10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media.

Afterward, the cultured NT unit or units can be washed and then placed in a suitable media contained in well plates which preferably contain a suitable confluent feeder layer. Suitable feeder layers include, by way of example, fibroblasts and epithelial cells. The NT units are cultured on the feeder layer until the NT units reach a size suitable for transferring to a recipient female, or for obtaining cells which can be used to produce cell colonies. Preferably, these NT units can be cultured until at least about 2 to 400 cells, more preferably about 4 to 128 cells, and most preferably at least about 50 cells.

Activated NT units can then be transferred (embryo transfers) to the oviduct of an female pigs. In one embodiment, the female pigs can be an estrus-synchronized recipient gilt. Crossbred gilts (large white/Duroc/Landrace) (280-400 lbs) can be used. The gilts can be synchronized as recipient animals by oral administration of 18-20 mg ReguMate (Altrenogest, Hoechst, Warren, N.J.) mixed into the feed. Regu-Mate can be fed for 14 consecutive days. One thousand units of Human Chorionic Gonadotropin (hCG, Intervet America, Millsboro, Del.) can then be administered i.m. about 105 h after the last Regu-Mate treatment. Embryo transfers of the can then be performed about 22-26 h after the hCG injection. In one embodiment, the pregnancy can be brought to term and result in the birth of live offspring. In another embodiment, the pregnancy can be 5 terminated early and embryonic cells can be harvested.

The methods for embryo transfer and recipient animal management in the present invention are standard procedures used in the embryo transfer industry. Synchronous transfers are important for success of the present invention, i.e., the stage of the NT embryo is in synchrony with the estrus cycle of the recipient female. See, for example, Siedel, G. E., Jr. “Critical review of embryo transfer procedures with cattle” in Fertilization and Embryonic Development in Vitro (1981) L. Mastroianni, Jr. and J. D. Biggers, ed., Plenum Press, New York, N.Y., page 323.

### VII. Porcine Animals, Organs, Tissues, Cells and Cell Lines

The present invention provides viable porcine in which both alleles of the porcine invariant chain gene have been inactivated. The invention also provides organs, tissues, and cells derived from such porcine, which are useful for xenotransplantation.

In one embodiment, the invention provides porcine organs, tissues and/or purified or substantially pure cells or cell lines obtained from pigs that lack any expression of functional invariant chain.

In one embodiment, the invention provides organs that are useful for xenotransplantation. Any porcine organ can be used, including, but not limited to: brain, heart, lungs, glands, brain, eye, stomach, spleen, pancreas, kidneys, liver, intestines, uterus, bladder, skin, hair, nails, ears, nose, mouth, lips, gums, teeth, tongue, salivary glands, tonsils, pharynx, esophagus, large intestine, small intestine, rectum, anus, pylorus, thyroid gland, thymus gland, suprarenal capsule, bones, cartilage, tendons, ligaments, skeletal muscles, smooth muscles, blood vessels, blood, spinal cord, trachea, ureters, urethra, hypothalamus, pituitary, adrenal glands, ovaries, oviducts, uterus, vagina, mammary glands, testes, seminal vesicles, penis, lymph, lymph nodes and lymph vessels.

In another embodiment, the invention provides tissues that are usefull for xenotransplantation. Any porcine tissue can be used, including, but not limited to: epithelium, connective tissue, blood, bone, cartilage, muscle, nerve, adenoid, adipose, areolar, bone, brown adipose, cancellous, muscle, cartaginous, cavernous, chondroid, chromaffin, dartoic, elastic, epithelial, fatty, fibrohyaline, fibrous, Gaingee, gelatinous, granulation, gut-associated lymphoid, Haller's vascular, hard hemopoietic, indifferent, interstitial, investing, islet, lymphatic, lymphoid, mesenchymal, mesonephric, mucous connective, multilocular adipose, myeloid, nasion soft, nephrogenic, nodal, osseous, osteogenic, osteoid, periapical, reticular, retiform, rubber, skeletal muscle, smooth muscle, and subcutaneous tissue.

In a further embodiment, the invention provides cells and cell lines from porcine animals that lack expression of functional alphα1,3GT. In one embodiment, these cells or cell lines can be used for xenotransplantation. Cells from any porcine tissue or organ can be used, including, but not limited to: epithelial cells, fibroblast cells, neural cells, keratinocytes, hematopoietic cells, melanocytes, chondrocytes, lymphocytes (B and T), macrophages, monocytes, mononuclear cells, cardiac muscle cells, other muscle cells, □hosphate cells, cumulus cells, epidermal cells, endothelial cells, Islets of Langerhans cells, pancreatic insulin secreting cells, pancreatic alpha-2 cells, pancreatic beta cells, pancreatic alpha-1 cells, blood cells, blood precursor cells, bone cells, bone precursor cells, neuronal stem cells, primordial stem cells., hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocy-tes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, leydig cells, peritubular cells, sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, embryonic stem cells, fibroblasts and fetal fibroblasts. In a specific embodiment, pancreatic cells, including, but not limited to, Islets of Langerhans cells, insulin secreting cells, 48 alpha-2 cells, beta cells, alpha-i cells from pigs that lack expression of functional alpha-1,3-GT are provided.

Nonviable derivatives include tisssues stripped of viable cells by enzymatic or chemical treatment these tissue derivatives can be further processed via crosslinking or other chemical treatments prior to use in transplantation. In a preferred embodiment, the derivatives include extracellular matrix derived from a variety of tissues, including skin, urinary, bladder or organ submucosal tissues. Also, tendons, joints and bones stripped of viable tissue to include heart valves and other nonviable tissues as medical devices are provided.

### Therapeutic Uses

The cells can be administered into a host in order in a wide variety of ways. Preferred modes of administration are parenteral, intraperitoneal, intravenous, intradermal, epidural, intraspinal, intrastemal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump, or via catheter. In one embodiment, the agent and carrier are administered in a slow release formulation such as a direct tissue injection or bolus, implant, microparticle, microsphere, nanoparticle or nanosphere.

Disorders that can be treated by infusion of the disclosed cells include, but are not limited to, diseases resulting from a failure of a dysfunction of normal blood cell production and maturation (i.e., aplastic anemia and hypoproliferative stem cell disorders); neoplastic, malignant diseases in the hematopoietic organs (e.g., leukemia and lymphomas); broad spectrum malignant solid tumors of non-hematopoietic origin; autoimmune conditions; and genetic disorders. Such disorders include, but are not limited to diseases resulting from a failure or dysfunction of normal blood cell production and maturation hyperproliferative stem cell disorders, including aplastic anemia, pancytopenia, agranulocytosis, thrombocytopenia, red cell aplasia, Blackfan Diamond syndrome, due to drugs, radiation, or infection, idiopathic; hematopoietic malignancies including acute lymphoblastic (lymphocytic) leukemia, chronic lymphocytic leukemia, acute myelogenous leukemia, chronic myelogenous, leukemia, acute malignant myelosclerosis, multiple myeloma, polycythemia vera, agnogenic myelometaplasia, Waldenstrom's macroglobulinemia, Hodgkin's lymphoma, non-Hodgkin's lymphoma; immunosuppression in patients with malignant, solid tumors including malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma, lymphonia; autoinimune diseases including rheumatoid arthritis, diabetes type 1, chronic hepatitis, multiple sclerosis, systemic lupus erythematosus; genetic (congenital) disorders including anemias, familial aplastic, Fanconi's syndrome, dihydrofolate reductase deficiencies, formamino transferase deficiency, Lesch-Nyhan syndrome, congenital dyserythropoietic syndrome IIV, Chwachmann-Diamond syndrome, dihydrofolate reductase deficiencies, forinamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose phosphate dehydrogenase) variants 1, 2, 3, pyruvate kinase deficiency, congenital erythropoietin sensitivity, deficiency, sickle cell disease and trait, thalassernia alpha, beta, gamma, met-hemoglobinemia, congenital disorders of immunity, severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital Leukocyte dysfunction syndromes; and others such as osteoporosis, myeloselerosis, acquired hemolytic anemias, acquired immunodeficiencies, infectious disorders causing primary or secondary immunodeficiencies, bacterial infections (e.g., Brucellosis, Listerosis, tuberculosis, leprosy), parasitic infections (e.g., malaria, Leishmaniasis), fungal infections, disorders involving disproportionsin lymphoid cell sets and impaired immune fimctions due to aging, phagocyte disorders, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, alpha lantirypsin deficiency, etc.

Diseases or pathologies include neurodegenerative diseases, hepatodegenerative diseases, nephrodegenerative disease, spinal cord injury, head trauma or surgery, viral infections that result in tissue, organ, or gland degeneration, and the like. Such neurodegenerative diseases include but are 1 0 not limited to, AIDS dementia complex; demyeliriating diseases, such as multiple sclerosis and acute transferase myelitis; extrapyramidal and cerebellar disorders, such as lesions of the ecorticospinal system; disorders of the basal ganglia or cerebellar disorders; hyperkinetic movement disorders, such as Huntington's Chorea and senile chorea; drug-induced movement disorders, such as those induced by drugs that block CNS dopamine receptors; hypokinetic movement disorders, such as Parkinson's disease; progressive supra-nucleo palsy; structural lesions of the cerebellum; spinocerebellar degenerations, such as spinal ataxia, Friedreich's ataxia, cerebellar cortical degenerations, multiple systems degenerations (Mencel, Dejerine Thomas, Shi-Drager, and Machado-Joseph), systermioc disorders, such as Rufsum's disease, abetalipoprotemia, ataxia, telangiectasia; and mitochondrial multisystem disorder; demyelinating core disorders, such as multiple sclerosis, acute transverse myelitis; and disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Demetia of Lewy body type; Parkinson's Disease, Wernicke-Korsakoff syndrome; chronic alcoholism; Creutzfeldt-Jakob disease; Subacute sclerosing panencephalitis hallefforden-Spatz disease; and Dementia pugilistica. See, e.g., Berkow et. aL, (eds.) (1987), The Merck Manual, (15′) ed.), Merck and Co., Rahway, N.J.

The following examples are offered by way of illustration and not by way of limitation.

### EXAMPLES

I. Cells and Tissues.

Porcine fetal tissues, including aorta, brain, and liver, were obtained from a local slaughterhouse. Samples to be used later for isolation of DNA or RNA were flash frozen in liquid nitrogen, whereas aortic tissue was treated with collagenase in phosphate-buffered saline and pig aortic endothelial cells (PAEC) were isolated. PAEC were maintained in Dulbecco's modified Eagle medium (DMEM, Gibco, Grand Island, N.Y.), 10,000 U of heparin sodium (Elkinns-Sinn, Inc., Cherry Hill, N.J.), 15 mg endothelium growth supplement (Collaborative Biomedical Products, Inc., Bedford, MA), L-glutamine, and penicillin-streptomycin. Culture flasks were kept loosely capped in a 37° C. incubator with an atmosphere of 5% CO₂.

II. Isolation of Nucleic Acids.

To isolate porcine genomic DNA, PAEC were grown to confluence in tissue culture flasks, trypsinized briefly at 37° C., and pelleted by centrifugation. High molecular weight porcine DNA was recovered using a standard protocol involving phenol-chloroform extraction, overnight incubation with RNase A, isopropanol precipitation, and spooling of precipitated DNA.

Total RNA was extracted from fetal tissue samples and cultured PAEC using Trizol reagent (Gibco) according to the manufacturer's instructions. For experiments in which polyadenylated (poly A⁺) RNA was used, poly A⁺ RNA was separated from total RNA using the Dynabeads mRNA Purification Kit (Dynal, Oslo, Norway) in accord with the protocol provided. Total yield of poly A⁺ RNA ranged from 1-5% of total RNA.

III. Genome Walking and Long PCR Amplification of Genomic DNA

A combination strategy of PCR-based methods was employed. Such PCR methods are well known in the art and described, for example, in PCR Technology, H. A. Erlich, ed., Stockton Press, London, 1989; PCR Protocols: A Guide to Methods and Applications, M. A. Innis, D. H. Gelfand, J. J. Sninsky, and T. J. White, eds., Academic Press, Inc., New York, 1990; and Ausubel et al.

5′- or 3′-RACE analyses. To identify the 5′ and 3′ ends of porcine invariant chain gene transcripts, 5′- and 3′-RACE procedures were performed using the Marathon cDNA Amplification Kit (Clontech) with PAEC poly A⁺ RNA as template. First strand cDNA synthesis from 1 μg of poly A⁺ RNA was accomplished using 20 U of AMV-RT and 1 pmol of the supplied cDNA Synthesis Primer by incubating at 48° C. for 2 hr. Second strand cDNA synthesis involved incubating the entire first strand reaction with a supplied enzyme cocktail composed of RNase H, Escherichia coli DNA polymerase I, and E. coli DNA ligase at 16° C. for 1.5 hr. After blunting of the double-stranded cDNA ends by T4 DNA polymerase, the supplied Marathon cDNA Adapters were ligated to an aliquot of purified, double-stranded cDNA. Dilution of the adapter-ligated product in 10 mM tricme-KOH/0 1 mM EDTA buffer provided with the kit readied the cDNA for PCR amplification. To obtain the 5′- and 3′-most sequences of porcine invariant chain transcripts, PCR reactions using the Marathon cDNA Adapter and Nested Adapter primers (mAP1 and mAP2) were paired with gene-specific and nested gene-specific primers (D1 and D2, described in Table 7) designed from an unidentified 157 base pair sequence (Table 6) contained in Katayama's clone D upstream of exon 4 of porcine alpha-1,3-GT gene as reported by Katayama A., et al, Glycoconj J. 1998 June;15(6):583-9, and used in a 3′RACE strategy. By this method, oligonucleotide primers based on the 157 base pair unidentified sequence described above are oriented in the 3′ and 5′ directions and are used to generate overlapping PCR fragments. These overlapping 3′- and 5′-end RACE products are combined to produce an intact full-length cDNA. This method is described in Innis et al., supra; and Frohman et al., Proc. Natl. Acad. Sci.,. 85:8998, 1988, and further described in U.S. Pat. No. 4,683,195.

Single major bands were obtained from two of the libraries, cloned, and subjected to sequence analysis. GenBank BLAST searches with those sequences revealed homology to exons 2,3, and 4 of the bovine invariant chain gene, as well as the intervening intron sequences (see GenBank Accession numbers D83961 and D83962). Based on close inspection and comparison of the sequences from porcine and bovine DNA, primer sets identified in Table 6 were designed for further 3′RACE (Table 8), and 5′RACE (Table 9). To facilitate amplification through the GC-rich portions of the 5′-untranslated sequences, the GC buffer supplied with the LA Taq enzyme was included in the PCR mix. 5′- and 3′-RACE products were subcloned and sequenced as described below.

Genome Walking analysis: To identify exon-intron boundaries, or 5′- or 3′-flanking region of the transcripts, porcine GenomeWalker™ libraries were constructed using a Universal GenomeWalkeer™ Library kit (Clontech, Palo Alto, Calif.). Briefly, five aliquots of porcine genomic DNA were separately digested with a single blunt-cutting restriction endonuclease (DraI, EcoRV, PvuII, ScaI, or StuI). After phenol-chloroform extraction, ethanol precipitation and resuspension of the restricted fragments, a portion of each digested aliquot was used in separate ligation reactions with the GenomeWalker adapters provided with the kit. This process created five “libraries” for use in the PCR-based cloning strategy of GenomeWalking. Primer pairs identified in Table 6 were used in combination in a genome walking strategy as identified in Table 10. Either eLON-Gase or TaKaRa LA Taq (Takara Shuzo Co., Ltd., Shiga, Japan) enzyme was used for PCR in all GenomeWalker experiments as well as for direct long PCR of genomic DNA. The thermal cycling conditions recommended by the manufacturer were employed in all GW-PCR experiments on a Perkin Elmer Gene Amp System 9600 or 9700 thermocycler.

Subcloning and sequencing of amplified products: PCR products amplified from genomic DNA, Gene Walker-PCR (Clontech), and 5′- or 3′-RACE were gel-purified using the Qiagen Gel Extraction Kit (Qiagen, Valencia, Calif.), if necessary, then subcloned into the pCR II vector provided with the Original TA Cloning Kit (Invitrogen, Carlsbad, Calif.). Plasmid DNA minipreps of pCR II-ligated inserts were prepared with the QIAprep Spin Miniprep Kit (Qiagen) as directed. Automated fluorescent sequencing of cloned inserts was performed using an ABI 377 Automated DNA Sequence Analyzer (Applied Biosystems, Inc., Foster City, Calif.) with either the dRhodamine or BigDye Terminator Cycle Sequencing Kits (Applied Biosystems) primed with T7 and SP6 promoter primers or primers designed from internal insert sequences described in Table 7 and Table 11)

Primer synthesis. All oligonucleotides used as primers in the various PCR-based methods were synthesized on an ABI 394 DNA Synthesizer (Applied Biosystems, Inc., Foster City, Calif.) using solid phase synthesis and phosphoramidite nucleoside chemistry, unless otherwise stated.

TABLE 6

5′-ACATTTAGTGATGACTTTTATATTTAGAATTAGCCAGCTGGACAAGC
TGACGGTCACCTCTCAGAACTTGCAGCTGGAGAGCTGCGGATGAAGCTTC
CCAAGCCCTCCAAGCCTTTGAGCAAGATGCGGGTTTCCGCACCCAGCTTC
TGCCGATCAG-3′

TABLE 7

PRIMERS USED IN 3′RACE, 5′RACE, GENOME
WALKER PCR, AND STANDARD PCR REACTIONS

Primer

| | |
|---|---|
| Name | Sequence |

| | |
|---|---|
| D1 | 5′-AGCCAGCTGGACAAGCTGACGGTCACCT-3′ |
| D2 | 5′ TGCAGCTGGAGAGCTGCGGATGAAGCTT-3′ |
| ifA | 5′-CTCTTTGAGAACTGGCTGCGTCAGTGGC-3′ |
| ifJ | 5′-ATGAGCAAGAACTCGCTGGAGGAGACAC-3′ |
| ihA | 5′-TGCACTTCATCCCATGGGCCTCTGGCA-3′ |
| ihB | 5′-TCCCTGGATACCTCACGTCTCTTCAGAAGG-3′ |
| ihP | 5′-TCTGAAGAGACGTGAGGTATCCAGGGAGGA-3′ |
| ihK | 5′-TGCCAGAGGCCCATGGGATGAAGTGCA-3′ |
| ifP | 5′-GTGTCTCCTCCAGCGAGTTCTTGCTCATTTC-3′ |
| ieK | 5′-ACCGTCCATGGTGTTCTTGAGGTGCTTTC-3′ |
| VR | 5′-CAGGAGCAGGTGCATCACGTGGTCCTG-3′ |
| VS | 5′-AAGCTTCATCCGCAGGCTCTCCAGCTGCA-3′ |
| VQ | 5′-GTTCTGAGAGGTGACCGTCAGCTTGTC-3′ |
| Vd | 5′-CCCAGCATGGGCAGCTGCTCATGGTTG-3′ |
| Ve | 5′-GAGATGAGGTCGCGCTGGTCCTCCATG-3′ |
| Vf | 5′-ACAGCAGGATCCGTAGCAGCAGCAGCAGCA-3′ |

TABLE 8
PRIMER SETS USED FOR 3′ RACE

| | | |
|---|---|---|
| D1 × D2 | ifA × ifJ | ihA × ihB |

TABLE 9
PRIMER SETS USED FOR 5′ RACE

| | | |
|---|---|---|
| ihP × ihK | ifP × VR | VS × VQ |

TABLE 10
PRIMER SETS USED FOR GENOME WALKING PCR

| | |
|---|---|
| D1 × D2 | for 3′ flanking region of exon 2 |
| Vd × Ve | for upstream region of exon 2 |
| ihP × ihK | for upstream region of exon 8 |
| ihA × ihB | for 3′ flanking region of exon 8 |

TABLE 11
PRIMER SETS FOR STANDARD PCR

| | |
|---|---|
| Vf × VS | for region between exon 1 and exon 2 |
| D2 × VR | for region between exon 2 and exon 4 |
| Vm × ieK | for region between exon 4 and exon 5 |
| ihA × ihP | for region between exon 7 and exon 8 |

Construction of Porcine Invariant Chain Homologous Recombination Targeting Vector

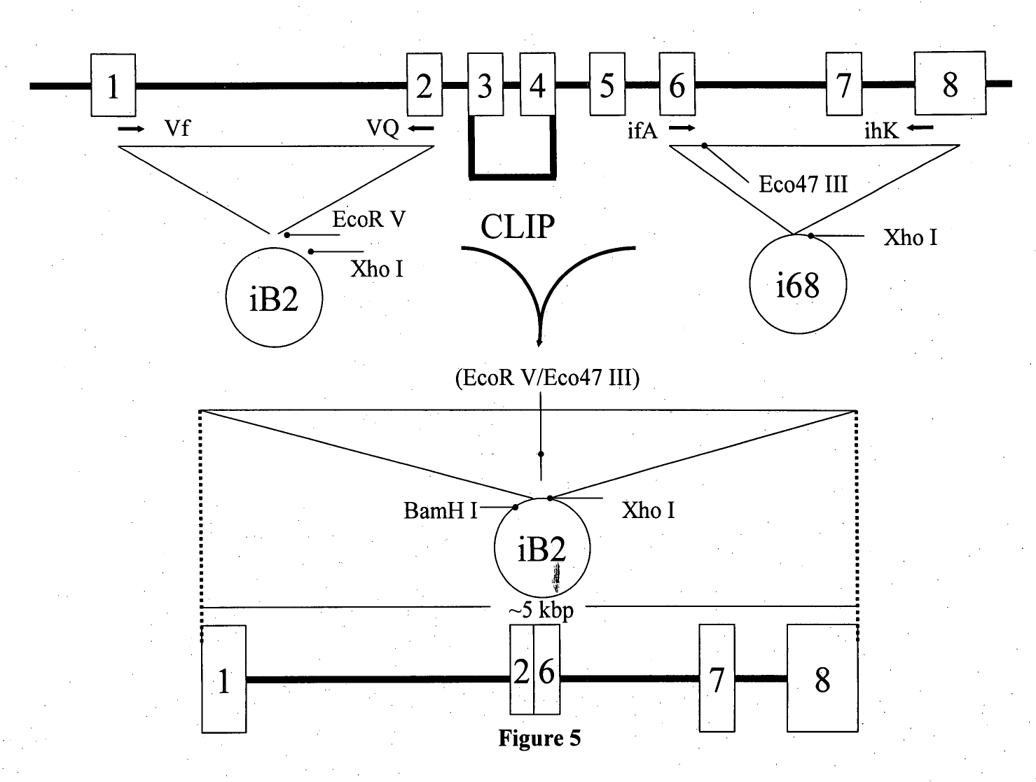

Invariant knock-out target vector: A vector targeting Exons 3, 4, and 5 (containing the CLIP peptide) of the porcine invariant chain gene for knockout can be constructed. In a first step, a portion of Exon 1, Intron 1, and Exon 2 is amplified by PCR for use as a 5′-arm of the targeting vector utilizing primers such as Vf (5′-ACAGCAGGATCCGTAGCAGCAGCAGCAGCA-3′), which introduces a BamHI. restriction site into the sequence, and VQ (5′-GTTCTGAGAGGTGACCGTCAGCTTGTC-3′) (see FIG. 5). The amplified PCR product can be inserted into a pCRII vector from Invitrogen after restriction enzyme digestion.

Exon 6, Intron 6, Exon 7, Intron 7, and a portion of Exon 8 can be amplified by PCR for use as a 3′-arm in the targeting vector utilizing primers such as ifA (5′-CTCTTTGAGAACTGGCTGCGTCAGTGGC-3′) and ihK (5′-TGCCAGAGGCCCATGGGATGAAGTGCA-3′) (see FIG. 5). The amplified PCR product can be inserted into a pCRII vector from Invitrogen. The vector containing the 5′ arm can be linearized by digestion with EcoRv and XhoI, and ligated with the 3′ fragment following digestion with Eco47 III and XhoI (see FIG. 4). The target vector, when homologously recombinated with the porcine invariant chain gene, can “knock-out” Exons 3, 4, and 5, as well as Introns 2, 3, 4, and 5.

Production of Porcine Invariant Chain Deficient Fetal Fibroblast Cells

Fetal fibroblast cells are isolated from 10 fetuses of the same pregnancy at day 33 of gestation. After removing the head and viscera, fetuses are washed with Hanks′ balanced salt solution (HBSS; Gibco-BRL, 1 5 Rockville, Md.), placed in 20 ml of HBSS, and diced with small surgical scissors. The tissue is pelleted and resuspended in 50-ml tubes with 40 ml of DMEM and 100 U/ml collagenase (Gibco-BRL) per fetus. Tubes are incubated for 40 min in a shaking water bath at 37 C. The digested tissue is allowed to settle for 3-4 min and the cell-rich supernatant is transferred to a new 50-ml tube and pelleted. The cells are then resuspended in 40 ml of DMEM containing 10% fetal calf serum (FCS), 1X nonessential amino acids, 1 mM sodium pyruvate and 2 ng/ml bFGF, and seeded into 10 cm. dishes. For transfections, 10 μg of linearized iB2 vector is introduced into 2 million cells using lipofectamine 2000 (Carlsbad, Calif.) following manufacturer's guidelines. Forty-eight hours after transfection, the transfected cells are seeded into 48-well plates at a density of 2,000 cells per well and grown to confluence. Following confluence, cells can be exposed to an anti-porcine invariant chain antibody, subsequently exposed to a FITC labeled secondary antibody, and separated via FACS sorting. Cells that do not bind with the anti-porcine invariant chain antibodies are further selected.

Selected cells are then reseeded, and grown to confluency. Once confluency is reached, several small aliquots are frozen back for future use, and the remainder are utilized for PCR and Southern Blot verification of homologous recombination. The putative targeted clones can be screened by PCR across the Exon 2/Exon 6 insert utilizing a primer complimentary to the Exon2/Exon 6 boundary sequence and a primer complimentary to a sequence outside the vector as the antisense primer. The PCR products can be analyzed by Southern Blotting using a probe to identify the positive clones by the presence of the expected band from the targeted allele.

Generation of Cloned Pigs Using Heterologous Invariant Chain Deficient Fetal Fibroblasts as Nuclear Donors

Preparation of cells for Nuclear Transfer: Donor cells are genetically manipulated to produce cells heterozygous for porcine invariant chain as described generally above. Nuclear transfer can be performed by methods that are well known in the art (see, e.g., Dai et al., Nature Biotechnology 20: 251255, 2002; and Polejaeva et al., Nature 407:86-90, 2000), using selected porcine fibroblasts as nuclear donors that are produced as described in detail hereinabove.

Oocytes can be isolated from synchronized super ovulated sexually mature Large-White X Landacre outcross gilts as described, for example, in I. Polejaeva et al. Nature 407: 505 (2000). Donor cells are synchronized in presumptive G0/G1 by serum starvation (0.5%) between 24 to 120 hours. Oocytes enucleation, nuclear transfer, electrofusion, and electroactivation can be performed as essentially described in, for example, A. C. Boquest et al., Biol. Reproduction 68: 1283 (2002). Reconstructed embryos can be cultured overnight and can be transferred to the oviducts of asynchronous (−1 day) recipients. Pregnancies can be confirmed and monitored by real-time ultrasound.

Breeding of heterozygous invariant chain single knockout (SKO) male and female pigs can be performed to establish a miniherd of double knockout (DKO) pigs.

Verification of Invariant Chain Deficient Pigs

Following breeding of the single knockout male and female pigs, verification of double knockout pigs is performed. Fibroblasts from the offspring are incubated with 1Ig of anti-porcine invariant chain antibody on ice for 30 minutes. FITC conjugated rabbit anti-mouse IgG is added to the cells and antibody binding indicating the presence or absence of porcine invariant chain, and thus, an indication of the presence or absence of active invariant chain, is detected by flow cytometry (FACSCalibur, Becton Dickenson, San Jose, Calif.).

This invention has been described with reference to its preferred embodiments. Variations and modifications of the invention, will be obvious to those skilled in the art from the foregoing detailed description of the invention. It is intended that all of these variations and modifications be included within the scope of this invention.

1. An isolated full length cDNA sequence encoding a porcine invariant chain protein.

2. The cDNA of claim 1 wherein the sequence is SEQ ID NO 1.

3. An isolated amino acid sequence encoding a porcine invariant chain protein.

4. A nucleic acid construct comprising a full length cDNA sequence encoding a porcine invariant chain protein.

5. The construct of claim 4, wherein the cDNA is SEQ ID NO 1.

6. The construct of claim 4, further comprising a promoter.

7. The construct of claim 4, further comprising a selectable marker.

8. The construct of claim 7, wherein the selectable marker is green fluorescent protein.

9. A transfected cell comprising the construct of any one of claims 4-8.

10. A cell expressing a porcine invariant chain protein.

11. The cell of claim 10 wherein the protein sequence is SEQ ID No 2.

12. An isolated nucleotide sequence homologous to at least a portion of SEQ ID No 1.

13. The nucleotide sequence of claim 12, wherein the sequence is at least 80% homologous to the nucleotide sequence of SEQ ID No 1.

14. The nucleotide sequence of claim 12, wherein the sequence is at least 90% homologous to the nucleotide sequence of SEQ ID No 1.

15. A nucleic acid construct comprising a nucleotide sequence homologous to at least a portion of SEQ ID No 1.

16. An isolated nucleotide sequence that hybridizes to SEQ ID No 1.

17. The nucleotide sequence of claim 16 that hybridizes under stringent conditions.

18. A vector comprising a nucleotide sequence of SEQ ID No 1.

19. A plasmid comprising a nucleotide sequence of SEQ ID No 1.

20. An isolated nucleotide comprising a nucleotide sequence of SEQ ID No 19.

21. An isolated nucleotide sequence selected from the group consisting of SEQ ID No 3, SEQ ID No 4, SEQ ID No. 5, SEQ ID No. 6, SEQ ID No. 7, SEQ ID No. 8, SEQ ID No. 9, SEQ ID No. 10, SEQ ID No. 11, SEQ ID No 12, SEQ ID No 13, SEQ ID No 14, SEQ ID No 15, SEQ ID No 16, SEQ ID No 17, and SEQ ID No 18.

22. A vector comprising a sequence of any of claims 20 and 21.

23. A nucleic acid construct comprising a sequence of any of claims 20 and 21.

24. A plasmid comprising a sequence of any of claims 20 and 21.

25. A nucleic acid construct comprising at least 17 contiguous nucleic acids of a sequence selected from the group comprising SEQ ID No 3, SEQ ID No 4, SEQ ID No. 5, SEQ ID No. 6, SEQ ID No. 7, SEQ ID No. 8, SEQ ID No. 9, SEQ ID No. 10, SEQ ID No. 11, SEQ ID No 12, SEQ ID No 13, SEQ ID No 14, SEQ ID No 15, SEQ ID No 16, SEQ ID No 17, and SEQ ID No 18.

26. A nucleic acid construct comprising at least 17 contiguous nucleic acids of SEQ ID No 19.

27. A nucleic acid construct comprising at least 50 contiguous nucleic acids of SEQ ID No. 19.

28. A nucleic acid construct comprising at least 200 contiguous nucleic acids of SEQ ID No.19.

29. A nucleic acid construct comprising at least 250 contiguous nucleic acids of SEQ ID No.19.

30. An isolated nucleotide comprising a sequence homologous to a nucleotide sequence of any of claims 20 to 21.

31. An isolated nucleotide that hybridizes to a nucleotide sequence of any of claims 20 to 21.

32. The nucleotide of claim 31 that hybridizes under stringent conditions.

33. A targeting vector comprising:
(a) a first nucleotide sequence comprising at least 17 contiguous nucleic acids homologous to SEQ ID No 19;
(b) a selectable marker gene; and
(c) a second nucleotide sequence comprising at least 17 contiguous nucleic acids homologous to SEQ ID No 19.

34. The targeting vector of claim 33 wherein the selectable marker is green fluorescent protein.

35. The targeting vector of claim 33 wherein the first nucleotide sequence represents the 5′ recombination arm.

36. The targeting vector of claim 33 wherein the second nucleotide sequence represents the 3′ recombination arm.

37. A cell transfected with the targeting vector of claim 33.

38. The cell of claim 37 wherein at least one allele of a porcine invariant chain gene has been rendered inactive via homologous recombination.

39. A porcine animal comprising the cell of claim 37.

40. The animal of claim 39 wherein at least one allele of a porcine invariant chain gene has been rendered inactive via homologous recombination.

41. An organ obtained from the animal of claim 40.

42. A tissue obtained from the animal of claim 40.

43. The organ of claim 42 wherein the organ is selected from the group consisting of heart, lung, kidney and liver.

44. A method to produce a genetically modified cells comprising:
(a) transfecting a porcine cell with the targeting vector of claim 33; and
(b) selecting a transfected cell in which at least one allele of a porcine invariant chain gene has been rendered inactive.

45. A method to produce a genetically modified animal comprising:
(a) transfecting a porcine cell with the targeting vector of claim 33;
(b) selecting a tranfected cell in which at least one allele of a porcine invariant chain gene has been rendered inactive (a nuclear donor cell);
(c) transferring the nucleus of the nuclear donor cell into an enucleated oocyte to produce an embryo; and
(d) allowing the embryo to develop into an animal.

46. An organ derived from the animal of claim 45.

47. The organ of claim 46 wherein the organ is selected from the group consisting of heart, lung, kidney and liver.

48. A tissue derived from the animal of claim 45.

49. The amino acid sequence of claim 3 wherein the sequence is SEQ ID NO 2.
